(19) United States
(12) Patent Application Publication
(10) Pub. No.: US 2023/0280348 A1
(43) Pub. Date: Sep. 7, 2023

(54) DIAGNOSIS OF CANCER

(71) Applicant: OXFORD UNIVERSITY INNOVATION LIMITED, Oxford (GB)

(72) Inventors: Fay PROBERT, Oxford (GB); James Robert LARKIN, Oxford (GB); Nicola SIBSON, Oxford (GB); Daniel ANTHONY, Oxford (GB); Timothy CLARIDGE, Oxford (GB)

(21) Appl. No.: 18/018,324

(22) PCT Filed: Jul. 29, 2021
(86) PCT No.: PCT/GB2021/051963

§ 371 Date: Jan. 27, 2023

(30) Foreign Application Priority Data
Jul. 29, 2020 (GB) 2011743.8

(51) Int. Cl. G01N 33/574; G01N 27/72

(52) U.S. Cl. CPC G01N 33/57488; G01N 27/72

(57) ABSTRACT

The present invention is directed to a method for diagnosing cancer, the method comprising: (a) comparing a concentration of one or more metabolite(s) present in a sample obtained from a subject with the concentration of the same one or more metabolite(s) in a reference standard, wherein the one or more metabolite(s) are selected from: a lipoprotein, myo-inositol, glucose, β-hydroxybutyrate, β-hydroxybutyric acid, an N-acetylated glycoprotein (NAC), threonine, lactate, lactic acid, acetate, and acetic acid; and (b) diagnosing cancer when: (i) the concentration of one or more metabolite(s) selected from: myo-inositol glucose, β-hydroxybutyrate, β-hydroxybutyric acid, NAC, and threonine is higher in the sample relative to the reference standard, wherein the reference standard is a non-cancer reference standard; and/or (ii) the concentration of one or more metabolite(s) selected from: a lipoprotein, lactate, lactic acid, acetate, and acetic acid is lower in the sample relative to the reference standard, wherein the reference standard is a non-cancer reference standard; and/or (iii) the concentration of one or more metabolite(s) selected from: myo-inositol, glucose, β-hydroxybutyrate, β-hydroxybutyric acid, NAC, and threonine is the same or higher in the sample relative to the reference standard, wherein the reference standard is a cancer reference standard; and/or (iv) the concentration of one or more metabolite(s) selected from: a lipoprotein, lactate, lactic acid, acetate, and acetic acid is the same or lower in the sample relative to the reference standard, wherein the reference standard is a cancer reference standard; or (c) not diagnosing cancer when: (i) the concentration of one or more metabolite(s) selected from: myo-inositol, glucose, β-hydroxybutyrate, β-hydroxybutyric acid, NAC, and threonine is the same or lower in the sample relative to the reference standard, wherein the reference standard is a non-cancer reference standard; and/or (ii) the concentration of one or more metabolite(s) selected from: a lipoprotein, lactate, lactic acid, acetate, and acetic acid is the same or higher in the sample relative to the reference standard, wherein the reference standard is a non-cancer reference standard; and/or (iii) the concentration of one or more metabolite(s) selected from: myo-inositol, glucose, β-hydroxybutyrate, β-hydroxybutyric acid, NAC, and threonine is lower in the sample relative to the reference standard, wherein the reference standard is a cancer reference standard; and/or (iv) the concentration of one or more metabolite(s) selected from: a lipoprotein, lactate, lactic acid, acetate, and acetic acid is higher in the sample relative to the reference standard, wherein the reference standard is a cancer reference standard. Methods for diagnosing a primary cancer or a secondary cancer are also provided.

The present invention relates to cancer and methods for diagnosing and/or treating the same.

Cancer is a serious ongoing public health concern accounting for 7.6 million of the 58 million deaths worldwide in 2005. Cancer incidence has since increased each year, with a prediction that it will account for 11.4 million deaths in 2030. In fact, worldwide, one person dies every 5 seconds as a result of cancer. It is well established that early treatment is more effective and improves patient outcomes, yet early diagnosis remains a significant challenge.

A major challenge for early detection of cancer is to identify those individuals with non-specific symptoms, also known as ‘silent killers’. Currently, there is no diagnostic pathway for such patients, which leads to severe delays in diagnosis during which symptoms worsen and the therapeutic window of opportunity is lost. Many of these patients eventually become emergency cases, for whom prognosis is typically very poor. There is, thus, a need for diagnostic methods that allow for early and/or reliable cancer diagnosis.

Presently, there is no diagnostic pathway for patients with non-specific signs, such as unexplained weight loss, severe unexplained fatigue, persistent nausea or appetite loss, new atypical pain, unexplained laboratory test finding, GP clinical suspicion of cancer or serious disease (GP ‘gut feeling’). Typically, the only option is a full body computerised tomography (CT) scan which is expensive and not routinely available in many cases. There is thus a need for low-cost and effective screening of patients with non-specific symptoms ensuring that patients are entered onto the correct diagnostic pathway, thereby reducing the number of full-body CT scans, and ensuring early diagnosis.

The present invention provides a solution to at least one of the problems described above.

The present inventors have surprisingly found that methods of the present invention allow for improved cancer diagnosis. Advantageously, the methods allow for diagnosis at an early stage based on a subject's metabolite concentration profile. Said early stage diagnosis allows for immediate treatment, thereby improving a subject's prognosis. The methods are also inexpensive to perform.

The methods of the invention also allow for differentiating between a primary and a secondary cancer. Said differentiation allows a clinician to determine an appropriate therapy/regimen, thereby improving a subject's prognosis.

Advantageously, the methods are particularly accurate and/or sensitive and/or specific. Thus, cancer can be reliably diagnosed (and/or differentiation between a primary and secondary cancer can be achieved) even when a subject presents with non-specific symptoms. In more detail, the methods of the invention reliably identify cancer in patients with ‘silent killer’ symptoms. The methods allow the correct cancer pathway to be identified for subjects with such non-specific symptoms/signs, ensuring appropriate treatment can be applied early when there is the greatest chance of response.

Cancer cells have a profound impact on the metabolic reactions throughout the body which are detectable in samples, such as blood, as a unique metabolite signature/profile. Techniques, such as NMR spectroscopy, allow for measurement of the relative concentrations of a large number of metabolites in samples quickly, cheaply, and reproducibly. These metabolites can then be used individually to diagnose cancer or in a multivariate manner (e.g. using the algorithm created by the multivariate analysis method described herein).

In a broad aspect, the invention provides a method for diagnosing cancer, the method comprising:
(a) comparing a concentration of one or more metabolite(s) present in a sample obtained from a subject with the concentration of the same one or more metabolite(s) in a reference standard, wherein the one or more metabolite(s) are selected from: glucose, β-hydroxybutyrate, β-hydroxybutyric acid, an N-acetylated glycoprotein (NAC), threonine, myo-inositol, a lipoprotein, lactate, lactic acid, acetate, acetic acid, and free choline; and
(b) diagnosing cancer or not diagnosing cancer based on the comparison.

In one aspect, the invention provides a method for diagnosing cancer, the method comprising:
(a) comparing a concentration of one or more metabolite(s) present in a sample obtained from a subject with the concentration of the same one or more metabolite(s) in a reference standard, wherein the one or more metabolite(s) are selected from: glucose, β-hydroxybutyrate, β-hydroxybutyric acid, NAC, threonine, myo-inositol, a lipoprotein, lactate, lactic acid, acetate, and acetic acid; and
(b) diagnosing cancer when:
   (i) the concentration of one or more metabolite(s) selected from: glucose, β-hydroxybutyrate, β-hydroxybutyric acid, NAC, threonine, and myo-inositol is higher in the sample relative to the reference standard, wherein the reference standard is a non-cancer reference standard; and/or
   (ii) the concentration of one or more metabolite(s) selected from: a lipoprotein, lactate, lactic acid, acetate, and acetic acid is lower in the sample relative to the reference standard, wherein the reference standard is a non-cancer reference standard; and/or
   (iii) the concentration of one or more metabolite(s) selected from: glucose, β-hydroxybutyrate, β-hydroxybutyric acid, NAC, threonine, and myo-inositol is the same or higher in the sample relative to the reference standard, wherein the reference standard is a cancer reference standard; and/or
   (iv) the concentration of one or more metabolite(s) selected from: a lipoprotein, lactate, lactic acid, acetate, and acetic acid is the same or lower in the sample relative to the reference standard, wherein the reference standard is a cancer reference standard.

In another aspect, the invention provides a method for diagnosing cancer, the method comprising:
(a) comparing a concentration of one or more metabolite(s) present in a sample obtained from a subject with the concentration of the same one or more metabolite(s) in a reference standard, wherein the one or more metabolite(s) are selected from: glucose, β-hydroxybutyrate, β-hydroxybutyric acid, NAC, threonine, myo-inositol, a lipoprotein, lactate, lactic acid, acetate, and acetic acid; and
(b) not diagnosing cancer when:
   (i) the concentration of one or more metabolite(s) selected from: glucose, β-hydroxybutyrate, β-hydroxybutyric acid, NAC, threonine, and myo-inositol is the same or lower (preferably the same) in the sample relative to the reference standard, wherein the reference standard is a non-cancer reference standard; and/or
   (ii) the concentration of one or more metabolite(s) selected from: a lipoprotein, lactate, lactic acid, acetate, and acetic acid is the same or higher (preferably the same) in the sample relative to the reference standard, wherein the reference standard is a non-cancer reference standard; and/or
   (iii) the concentration of one or more metabolite(s) selected from: glucose, β-hydroxybutyrate, β-hydroxybutyric acid, NAC, threonine, and myo-inositol is lower in the sample relative to the reference standard, wherein the reference standard is a cancer reference standard; and/or
   (iv) the concentration of one or more metabolite(s) selected from: a lipoprotein, lactate, lactic acid, acetate, and acetic acid is higher in the sample relative to the reference standard, wherein the reference standard is a cancer reference standard.

In a preferred embodiment, the invention provides a method for diagnosing cancer, the method comprising:
(a) comparing a concentration of one or more metabolite(s) present in a sample obtained from a subject with the concentration of the same one or more metabolite(s) in a reference standard, wherein the one or more metabolite(s) are selected from: glucose, β-hydroxybutyrate, β-hydroxybutyric acid, NAC, threonine, myo-inositol, a lipoprotein, lactate, lactic acid, acetate, and acetic acid; and
(b) diagnosing cancer when:
   (i) the concentration of one or more metabolite(s) selected from: glucose, β-hydroxybutyrate, β-hydroxybutyric acid, NAC, threonine, and myo-inositol is higher in the sample relative to the reference standard, wherein the reference standard is a non-cancer reference standard; and/or
   (ii) the concentration of one or more metabolite(s) selected from: a lipoprotein, lactate, lactic acid, acetate, and acetic acid is lower in the sample relative to the reference standard, wherein the reference standard is a non-cancer reference standard; or
(c) not diagnosing cancer when:
   (i) the concentration of one or more metabolite(s) selected from: glucose, β-hydroxybutyrate, β-hydroxybutyric acid, NAC, threonine, and myo-inositol is the same or lower (preferably the same) in the sample relative to the reference standard, wherein the reference standard is a non-cancer reference standard; and/or
   (ii) the concentration of one or more metabolite(s) selected from: a lipoprotein, lactate, lactic acid, acetate, and acetic acid is the same or higher (preferably the same) in the sample relative to the reference standard, wherein the reference standard is a non-cancer reference standard.

In one aspect, the invention provides a method for diagnosing cancer, the method comprising
(i) comparing a concentration of one or more metabolite(s) present in a sample obtained from a subject with the concentration of the same one or more metabolite(s) in a reference standard, wherein the one or more metabolite(s) are selected from: glucose, β-hydroxybutyrate, β-hydroxybutyric acid, NAC, threonine, myo-inositol, a lipoprotein, lactate, lactic acid, acetate, and acetic acid; and
(ii) identifying a concentration difference for the one or more metabolite(s) in the sample relative to the reference standard;
   wherein said concentration difference correlates with the presence or absence of cancer.

In a broad aspect, the invention provides a method for diagnosing cancer, the method comprising:
(a) comparing a ¹H-NMR spectrum of a sample obtained from a subject with a ¹H-NMR reference standard; and
(b) diagnosing cancer or not diagnosing cancer based on the comparison.

In a related broad aspect, the invention provides a method for diagnosing cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm; and
(b) diagnosing cancer or not diagnosing cancer based on the comparison.

In one aspect, the invention provides a method for diagnosing cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm; and
(b) diagnosing cancer when:
   (i) the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm is higher in the sample relative to the reference standard, wherein the reference standard is a non-cancer reference standard; and/or
   (ii) the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is lower in the sample relative to the reference standard, wherein the reference standard is a non-cancer reference standard; and/or
   (iii) the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm is the same or higher in the sample relative to the reference standard, wherein the reference standard is a cancer reference standard; and/or
   (iv) the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is the same or lower in the sample relative to the reference standard, wherein the reference standard is a cancer reference standard.

In one aspect, the invention provides a method for diagnosing cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm; and
(b) not diagnosing cancer when:
   (i) the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm is the same or lower in the sample relative to the reference standard, wherein the reference standard is a non-cancer reference standard; and/or
   (ii) the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is the same or higher in the sample relative to the reference standard, wherein the reference standard is a non-cancer reference standard; and/or
   (iii) the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm is lower in the sample relative to the reference standard, wherein the reference standard is a cancer reference standard; and/or
   (iv) the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is higher in the sample relative to the reference standard, wherein the reference standard is a cancer reference standard.

In a preferred embodiment, the invention provides a method for diagnosing cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm; and
(b) diagnosing cancer when:
   (i) the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm is higher in the sample relative to the reference standard, wherein the reference standard is a non-cancer reference standard; and/or
   (ii) the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is lower in the sample relative to the reference standard, wherein the reference standard is a non-cancer reference standard; or
(c) not diagnosing cancer when:
   (i) the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm is the same or lower in the sample relative to the reference standard, wherein the reference standard is a non-cancer reference standard; and/or
   (ii) the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is the same or higher in the sample relative to the reference standard, wherein the reference standard is a non-cancer reference standard.

In one aspect, the invention provides a method for diagnosing cancer, the method comprising
(i) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm; and
(ii) identifying a difference in intensity of the one or more chemical shift region(s) of the ¹H-NMR spectrum of the sample obtained from the subject relative to the reference standard;
   wherein said difference in intensity correlates with the presence or absence of cancer.

In a broad aspect, the invention provides a method for diagnosing a primary cancer or a secondary cancer, the method comprising:
(a) comparing a concentration of one or more metabolite(s) comprised in a sample obtained from a subject with the concentration of the same one or more metabolite(s) in a reference standard (preferably a cancer reference standard), wherein the one or more metabolite(s) are selected from: a lipoprotein, β-hydroxybutyrate, β-hydroxybutyric acid, NAC, threonine, myo-inositol, glucose, lactate, lactic acid, acetate, acetic acid, and free choline; and
(b) diagnosing a primary cancer or a secondary cancer based on the comparison.

In one aspect, the invention provides a method for diagnosing a primary cancer or a secondary cancer, the method comprising:
(a) comparing a concentration of one or more metabolite(s) comprised in a sample obtained from a subject with the concentration of the same one or more metabolite(s) in a reference standard, wherein the one or more metabolite(s) are selected from: a lipoprotein, β-hydroxybutyrate, β-hydroxybutyric acid, NAC, threonine, myo-inositol, glucose, lactate, lactic acid, acetate, acetic acid, and free choline; and
(b) diagnosing a secondary cancer when:
   (i) the concentration of one or more metabolite(s) selected from: a lipoprotein, β-hydroxybutyrate, β-hydroxybutyric acid, lactate, lactic acid, acetate, acetic acid, and free choline is higher in the sample relative to the reference standard, wherein the reference standard is a primary cancer reference standard; and/or
   (ii) the concentration of one or more metabolite(s) selected from: NAC, threonine, myo-inositol, and glucose is lower in the sample relative to the reference standard, wherein the reference standard is a primary cancer reference standard; and/or
   (iii) the concentration of one or more metabolite(s) selected from: a lipoprotein, β-hydroxybutyrate, β-hydroxybutyric acid, lactate, lactic acid, acetate, acetic acid, and free choline is the same or higher in the sample relative to the reference standard, wherein the reference standard is a secondary cancer reference standard; and/or
   (iv) the concentration of one or more metabolite(s) selected from: NAC, threonine, myo-inositol, and glucose is the same or lower in the sample relative to the reference standard, wherein the reference standard is a secondary cancer; or
(c) diagnosing a primary cancer when:
   (i) the concentration of one or more metabolite(s) selected from: a lipoprotein, β-hydroxybutyrate, β-hydroxybutyric acid, lactate, lactic acid, acetate, acetic acid, and free choline is the same or lower in the sample relative to the reference standard, wherein the reference standard is a primary cancer reference standard; and/or
   (ii) the concentration of one or more metabolite(s) selected from: NAC, threonine, myo-inositol, and glucose is the same or higher in the sample relative to the reference standard, wherein the reference standard is primary cancer reference standard; and/or
   (iii) the concentration of one or more metabolite(s) selected from: a lipoprotein, β-hydroxybutyrate, β-hydroxybutyric acid, lactate, lactic acid, acetate, acetic acid, and free choline is lower in the sample relative to the reference standard, wherein the reference standard is a secondary cancer reference standard; and/or
   (iv) the concentration of one or more metabolite(s) selected from: NAC, threonine, myo-inositol, and glucose is higher in the sample relative to the reference standard, wherein the reference standard is a secondary cancer reference standard.

In one aspect, the invention provides a method for diagnosing a primary cancer or a secondary cancer, the method comprising
(i) comparing a concentration of one or more metabolite(s) present in a sample obtained from a subject with the concentration of the same one or more metabolite(s) in a reference standard, wherein the one or more metabolite(s) are selected from: a lipoprotein, β-hydroxybutyrate, β-hydroxybutyric acid, NAC, threonine, myo-inositol, glucose, lactate, lactic acid, acetate, acetic acid, and free choline; and
(ii) identifying a concentration difference for the one or more metabolite(s) in the sample relative to the reference standard;
   wherein said concentration difference correlates with the presence of a primary cancer or a secondary cancer.

In a broad aspect, the invention provides a method for diagnosing a primary cancer or a secondary cancer, the method comprising:
(a) comparing a ¹H-NMR spectrum of a sample obtained from a subject with a ¹H-NMR reference standard (preferably a cancer reference standard); and
(b) diagnosing a primary cancer or a secondary cancer based on the comparison.

In a broad aspect, the invention provides a method for diagnosing a primary cancer or a secondary cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm; and
(b) diagnosing a primary cancer or a secondary cancer based on the comparison.

In one aspect, the invention provides a method for diagnosing a primary cancer or a secondary cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm; and
(b) diagnosing a secondary cancer when:
   (i) the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is higher in the sample relative to the reference standard, wherein the reference standard is a primary cancer reference standard; and/or
   (ii) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, and 3.17-3.95 ppm, 4.63-4.66 ppm, and 5.22-5.25 ppm is lower in the sample relative to the reference standard, wherein the reference standard is a primary cancer reference standard; and/or
   (iii) the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or higher in the sample relative to the reference standard, wherein the reference standard is a secondary cancer reference standard; and/or
   (iv) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or lower in the sample relative to the reference standard, wherein the reference standard is a secondary cancer; or
(c) diagnosing a primary cancer when:
   (i) the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or lower in the sample relative to the reference standard, wherein the reference standard is a primary cancer reference standard; and/or
   (ii) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or higher in the sample relative to the reference standard, wherein the reference standard is primary cancer reference standard; and/or
   (iii) the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is lower in the sample relative to the reference standard, wherein the reference standard is a secondary cancer reference standard; and/or
   (iv) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is higher in the sample relative to the reference standard, wherein the reference standard is a secondary cancer reference standard.

In one aspect, the invention provides a method for primary cancer or a secondary cancer, the method comprising
(i) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm; and
(ii) identifying a difference in intensity of the one or more chemical shift region(s) of the ¹H-NMR spectrum of the sample obtained from the subject relative to the reference standard;
   wherein said difference in intensity correlates with the presence of a primary cancer or a secondary cancer.

In one aspect, the invention provides a method, the method comprising:
(a) providing a sample obtained from a subject; and
(b) assaying the sample for a concentration of one or more metabolite(s) selected from: a lipoprotein, β-hydroxybutyrate, β-hydroxybutyric acid, NAC, threonine, myo-inositol, glucose, lactate, lactic acid, acetate, acetic acid, and free choline.

In one aspect, the invention provides a method, the method comprising:
(a) providing a sample obtained from a subject; and
(b) assaying the sample for an intensity of one or more chemical shift region(s) of a ¹H-NMR spectrum, wherein the one or more chemical shift region(s) are selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

A cancer diagnosed by a method of the invention may be a primary cancer or a secondary cancer.

A “primary cancer” as used herein refers to a localised cancer. In one embodiment, when a primary cancer is diagnosed, a subject does not have any secondary cancers. In a method of the invention, preferably when a primary cancer is diagnosed, the subject may not have metastatic cancer and may have a primary cancer only. A primary cancer is a cancer that has not arisen due to metastasis.

A “secondary cancer” as used herein refers to a metastatic cancer that is derived from a primary cancer. While a secondary cancer is typically of the same tissue type as a primary cancer, said secondary cancer may be genotypically and/or phenotypically different to the primary cancer. Such differences may arise due to genomic instability and accumulated mutations of said secondary cancer. Thus, when a secondary cancer is diagnosed, the subject preferably also has a primary cancer. In other words, diagnosing a secondary cancer means diagnosing metastatic disease.

Reference to a “cancer” herein may also encompass any tumour. Preferably, the term “cancer” as used herein encompasses a tumour that is a premalignant or malignant tumour and not a benign tumour.

A method of the invention may comprise a step of measuring a concentration of one or more metabolite(s) present in a sample obtained from a subject.

A method of the invention may comprise a step of obtaining a ¹H-NMR spectrum of a sample obtained from a subject.

The concentrations of the metabolites in a sample can be measured using any suitable technique known in the art. By way of example, the following techniques may be used alone or in combination to detect and quantify molecules in solution, and are thus suitable for determining metabolite concentrations: Nuclear Magnetic Resonance (NMR) spectroscopy, mass spectrometry, gas chromatography, ultraviolet (UV) spectrometry (for example in combination with high-performance liquid chromatography [HPLC] as HPLC-UV), infrared spectroscopy, and a biochemical assay. A metabolite is preferably identified using NMR, more preferably ¹H-NMR. The biochemical assay may be an enzymatic assay.

In one embodiment, the concentration of one or more metabolites is determined using NMR spectroscopy. In one embodiment, the concentration of one or more metabolites is determined using mass spectrometry. In one embodiment, the concentration of one or more metabolites is determined using HPLC-UV. In one embodiment, the concentration of one or more metabolites is determined using infrared spectroscopy.

The concentration of a metabolite in a sample can be expressed in a number of different ways, for example as a molar concentration (number of moles of metabolite per unit volume of sample) or a mass concentration (mass of metabolite per unit volume of sample). Alternatively, the concentration of a metabolite can be expressed as parts per million (ppm) or parts per billion (ppb). Such ways of expressing the concentration of a molecule in solution are known in the art. In some embodiments, a concentration of a metabolite may be expressed relative to a standard or to another metabolite within the sample. For example, when techniques such as NMR are employed a concentration may be expressed as a relative spectral intensity.

Thus, in one embodiment, the concentration of a metabolite in a sample is the molar concentration of said metabolite. In one embodiment, the concentration of a metabolite in a sample is the mass concentration of said metabolite.

The concentration of a metabolite in a sample may be expressed in absolute terms, for example as an absolute molar concentration or absolute mass concentration. Alternatively, the concentration of a metabolite in a sample can be expressed by comparison to the concentration of a different metabolite in the same sample (i.e. in relative terms). By way of example, the concentration of a metabolite in the sample can be normalised by comparison to the concentration of a different reference metabolite within the same sample.

The methods described herein are particularly sensitive and allow for accurate and/or sensitive and/or specific determination and/or diagnosis when using only one metabolite. Notably, even where the concentration of a metabolite has not been found to be statistically-significantly changed when compared to a reference standard, said metabolite has utility in a method of the invention, especially where used in combination with a further metabolite and/or when compared to multiple reference standards.

In one embodiment, a metabolite for use in the invention is a lipoprotein. A lipoprotein may be a very low density lipoprotein (VLDL), a low density lipoprotein (LDL) or a high density lipoprotein (HDL). In some embodiments, the methods employs the use of at least two of: a VLDL, a LDL, and an HDL.

A lipoprotein may be detected, and/or its concentration measured, by detecting a chemical group of the lipoprotein, for example a —CH₃ group of a lipoprotein. When using NMR, certain chemical shift ranges are characteristic of such groups of the various density lipoproteins, as described below.

In one embodiment, a method utilises a —CH₃ group of an HDL and/or LDL. A ¹H-NMR chemical shift range of 0.80-0.86 ppm may be characteristic of a —CH₃ group of an HDL and/or LDL.

In one embodiment, a method utilises a —CH₃ group of a VLDL. A ¹H-NMR chemical shift range of 0.86-0.92 ppm may be characteristic of a —CH₃ group of a VLDL.

In one embodiment, a method utilises a —(CH₂)ₙ group of a VLDL. A ¹H-NMR chemical shift range of 1.30-1.39 ppm may be characteristic of a —(CH₂)ₙ group of a VLDL.

In one embodiment, a method utilises a —(CH₂)ₙ group of an HDL and/or LDL. A ¹H-NMR chemical shift range of 1.15-1.30 ppm may be characteristic of a —(CH₂)ₙ group of an HDL and/or LDL.

In one embodiment, a method utilises a βH₂ group of a lipoprotein. A ¹H-NMR chemical shift range of 1.53-1.61 ppm may be characteristic of a βH₂ group of a lipoprotein.

In one embodiment, a method utilises an αCH₂ group of a lipoprotein. A ¹H-NMR chemical shift range of 2.20-2.26 ppm may be characteristic of an αCH₂ group of a lipoprotein.

In one embodiment, a method utilises an —N(CH₃)₃ group of a lipoprotein. A ¹H-NMR chemical shift range of 3.17-3.31 ppm may be characteristic of an —N(CH₃)₃ group of a lipoprotein.

In one embodiment, a metabolite for use in the invention is free choline. Said metabolite may be defined via a ¹H-NMR chemical shift range of 3.17-3.31 ppm.

In one embodiment, a metabolite for use in the invention is β-hydroxybutyrate and/or β-hydroxybutyric acid. Said metabolites may be defined via one or more ¹H-NMR chemical shift range(s) of 1.19-1.21 ppm and/or 2.27-2.45 ppm. Preferably, said metabolites may be defined via ¹H-NMR chemical shift ranges of 1.19-1.21 ppm and 2.27-2.45 ppm.

In one embodiment, a metabolite for use in the invention is lactate and/or lactic acid. Said metabolites may be defined via one or more ¹H-NMR chemical shift range(s) of 1.31-1.35 ppm and/or 4.08-4.14 ppm. Preferably, said metabolites may be defined via ¹H-NMR chemical shift ranges of 1.31-1.35 ppm and 4.08-4.14 ppm.

In one embodiment, a metabolite for use in the invention is acetate and/or acetic acid. Said metabolites may be defined via a ¹H-NMR chemical shift range of 1.88-1.93 ppm.

In one embodiment, a metabolite for use in the invention is an N-acetylated glycoprotein (NAC). Said metabolite may be defined via a ¹H-NMR chemical shift range of 1.93-2.10 ppm.

In one embodiment, a metabolite for use in the invention is citrate and/or citric acid. Said metabolites may be defined via a ¹H-NMR chemical shift range of 2.51-2.70 ppm.

In one embodiment, a metabolite for use in the invention is threonine. Said metabolite may be defined via one or more ¹H-NMR chemical shift range(s) of 3.57-3.59 ppm, 1.30-1.35 ppm, and/or 4.06-4.3 ppm. Preferably, said metabolite may be defined via ¹H-NMR chemical shift ranges of 3.57-3.59 ppm, 1.30-1.35 ppm, and 4.06-4.3 ppm.

In one embodiment, a metabolite for use in the invention is myo-inositol. Said metabolite may be defined via one or more ¹H-NMR chemical shift range(s) of 3.63-3.65 ppm, 3.53-3.58 ppm, 3.93-3.98 ppm, and/or 3.25-3.29 ppm. Preferably, said metabolite may be defined via ¹H-NMR chemical shift ranges of 3.63-3.65 ppm, 3.53-3.58 ppm, 3.93-3.98 ppm, and 3.25-3.29 ppm.

In one embodiment, a metabolite for use in the invention is glucose. Said metabolite may be defined via one or more ¹H-NMR chemical shift range(s) of 3.17-3.95 ppm, 4.63-4.66 ppm, and/or 5.22-5.25 ppm. Preferably, said metabolite may be defined via ¹H-NMR chemical shift ranges of 3.17-3.95 ppm, 4.63-4.66 ppm, and 5.22-5.25 ppm.

In one aspect, the metabolites herein may instead be referred to by their ¹H-NMR chemical shift range(s), as described above.

Preferably, wherein the method is a method for diagnosing primary cancer or secondary cancer, it is preferred that a metabolite employed is a lipoprotein (more preferably VLDL).

In some embodiments, more than one metabolite may be employed, i.e. a plurality of metabolites may be employed. In a preferred embodiment, at least 2 metabolites are employed in a method described herein.

The term “one or more” when used in the context of a metabolite described herein may mean at least 2, 3, 4, 5, 6, 7, 8, 9, 10, 11, 12 or 13 metabolites. When carrying out a method herein, it is preferred that those metabolites that are highest ranked in Table 4 are used, for example, where 2 metabolites are used, it is preferred that these are the 2 highest ranking metabolites. Preferably, wherein the method is a method for diagnosing cancer, it is preferred that at least glucose and lipoprotein (more preferably VLDL) are employed.

In one embodiment, the invention utilizes two or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, two or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes three or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, three or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes four or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, four or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes five or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, five or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes six or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, six or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes seven or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, seven or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes eight or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, eight or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes nine or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, nine or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes ten or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, ten or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes eleven or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, eleven or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes twelve or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, twelve or more metabolites selected from: glucose, β-hydroxybutyrate, β-hydroxybutyric acid, NAC, threonine, myo-inositol, a lipoprotein, lactate, lactic acid, acetate, acetic acid, and free choline.

In one embodiment, the invention utilizes thirteen or more metabolites selected from: glucose, β-hydroxybutyrate, β-hydroxybutyric acid, NAC, threonine, myo-inositol, a lipoprotein, lactate, lactic acid, acetate, acetic acid, citrate, citric acid, and free choline. Preferably, all of the following metabolites: glucose, β-hydroxybutyrate, β-hydroxybutyric acid, NAC, threonine, myo-inositol, a lipoprotein, lactate, lactic acid, acetate, acetic acid, and free choline.

In a particularly preferred embodiment, the invention utilizes all of the following metabolites: glucose, β-hydroxybutyrate, β-hydroxybutyric acid, NAC, threonine, myo-inositol, a lipoprotein, lactate, lactic acid, acetate, acetic acid, citrate, citric acid, and free choline.

Methods of the present invention are also based on the identification of chemical shift regions of ¹H-NMR spectra that allow for accurate and/or sensitive and/or specific diagnosis of cancer, a primary cancer, and/or a secondary cancer. By way of background, carrying out ¹H-NMR produces a spectrum, as is known in the art. The spectrum can be characterised according to the chemical shift positions (in ppm), which define peak positions, and the intensity of the peaks. Intensity (i.e. peak/spectral intensity) corresponds to the concentration of a chemical (e.g. a metabolite) present in a sample. Intensity may be determined by any method known in the art, such as determining an area under the peak. The Examples herein define a particularly preferred method for carrying out ¹H-NMR to produce a spectrum for use in the present invention.

The chemical shifts quoted herein may be considered to encompass a value that deviates from the quoted value by ±0.01 ppm, preferably a value that deviates from the quoted value by less than ±0.01 ppm, more preferably by 0 ppm.

In one embodiment, a suitable volume of a sample is diluted with an appropriate buffer (preferably having a pH meter reading of 7.4) and solvent. Preferably, the sample comprises D₂O. Preferably, a suitable volume (e.g. 150 μl) of a sample is diluted with (e.g. 450 μl) sodium phosphate buffer prepared in D₂O (pH meter reading of 7.4).

Said samples may be processed to remove any precipitate prior to carrying out NMR.

Preferably, the chemical shift regions quoted herein are reported relative to lactate —CH₃ referenced at 1.33 ppm.

Preferably, the ¹H-NMR is carried out on samples at 298K.

Most preferably, the chemical shift regions herein have been defined by carrying out “the ¹H-NMR assay” described herein. “The ¹H-NMR assay” comprises the following steps:
(a) diluting 150 μL of sample with 450 μL of 75 mM sodium phosphate buffer prepared in D₂O (pH meter reading of 7.4);
(b) centrifuging said diluted sample at 16,000×g for 3 minutes to remove any precipitate;
(c) transferring the supernatant to a 5 mm NMR tube;
(d) obtaining an NMR spectrum of the sample, wherein the spectrum is obtained using a 1D NOESY presaturation scheme for attenuation of the water resonance with a 2 s presaturation using a 700-MHz NMR spectrometer equipped with a helium-cooled cryogenic probe (e.g. a 700-MHz Bruker AVIII spectrometer operating at 16.4T equipped with a ¹H (¹³C/¹⁵N) TCI cryoprobe) at a sample temperature of 298K;
(e) zero-filling resulting free induction decays (FIDs) by a factor of 2 and multiplying by an exponential function corresponding to 0.30 Hz line broadening prior to Fourier transformation;
(f) phasing and baseline correcting (using a 3^{rd} degree polynomial) the spectrum; and
(g) referencing chemical shifts to the lactate-CH₃ doublet resonance at δ=1.33 ppm; and
(h) optionally, when the sample is a serum or plasma sample, in addition to the 1D NOESY presaturation scheme, a spin-echo Carr-Purcell-Meiboom-Gill (CPMG) sequence with a T interval of 400 μs, 80 loops, 32 data collections, an acquisition time of 1.5 s, and a relaxation delay of 2 s may be used to supress broad signals arising from large molecular weight blood components;
(i) optionally, for blood plasma and blood serum spectra, the regions between 0.20-4.70 ppm and 5.00-9.60 ppm may be divided in to 0.01 ppm width ‘buckets’. For urine spectra, the regions between 0.20-4.70 ppm and 5.00-5.70 and 5.96-9.60 ppm may be divided in to 0.01 ppm width ‘buckets’; and
(j) optionally the absolute value of the integral of each spectral bucket may be Pareto scaled; and
(k) optionally, resonances may be assigned by reference to literature values [Anal Biochem 325:260-272, J Pharm Biomed Anal 33:1103-1115] and the Human Metabolome Database [Nucleic Acids Res 41:D801-807. doi:10.1093/nar/gks1065, Nucleic Acids Res 37:D603-610. doi:10.1093/nar/gkn810, Nucleic Acids Res 35:D521-526. doi:10.1093/nar/gkl923] and further confirmed by inspection of the 2D spectra, spiking of known compounds, and 1D-TOCSY spectra.

In one embodiment, the invention encompasses ¹H-NMR techniques carried out under conditions other than those defined herein. In the unlikely event that said techniques lead to one or more different chemical shift region(s), said different chemical shift region(s) are encompassed by the present invention so long as the different chemical shift region(s) correspond to the chemical shift region(s) presented herein when carried out using “the ¹H-NMR assay” described herein.

A method of the invention may utilize one or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

The methods comprising the use of chemical shift regions of ¹H-NMR spectra are accurate and/or sensitive and/or specific when using only one chemical shift region. Notably, even where the intensity of a chemical shift region has not been found to be statistically-significantly changed when compared to a reference standard, said chemical shift region has utility in a method of the invention, especially where used in combination with a further chemical shift region and/or when compared to multiple reference standards.

In some embodiments, more than one chemical shift region may be employed, i.e. a plurality of chemical shift regions may be employed. In a preferred embodiment, at least 2 chemical shift regions are employed in a method described herein.

The term “one or more” when used in the context of a chemical shift region described herein may mean at least 2, 3, 4, 5, 6, 7, 8, 9, 10, 11, 12, 13, 14 or 15 chemical shift regions. When carrying out a method herein, it is preferred that those chemical shift regions that are highest ranked in Table 4 are used, for example, where 2 chemical shift regions are used, it is preferred that these are the 2 highest ranking chemical shift regions. Preferably, wherein the method is a method for diagnosing cancer, it is preferred that at least two chemical shift regions employed are selected from 3.17-3.95 ppm, 4.63-4.66 ppm, 5.22-5.25 ppm, and 1.30-1.39 ppm, more preferably all of 3.17-3.95 ppm, 4.63-4.66 ppm, 5.22-5.25 ppm, and 1.30-1.39 ppm are employed. Preferably, wherein the method is a method for diagnosing primary cancer or secondary cancer, it is preferred that at least two chemical shift regions employed are 0.86-0.92 ppm and 1.30-1.39 ppm.

Thus, in one embodiment, the invention utilizes two or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, 3.17-3.31 ppm, and 2.51-2.70 ppm. Preferably, two or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

In one embodiment, the invention utilizes three or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, 3.17-3.31 ppm, and 2.51-2.70 ppm. Preferably, three or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

In one embodiment, the invention utilizes four or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, 3.17-3.31 ppm, and 2.51-2.70 ppm. Preferably, four or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

In one embodiment, the invention utilizes five or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, 3.17-3.31 ppm, and 2.51-2.70 ppm. Preferably, five or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

In one embodiment, the invention utilizes ten or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, 3.17-3.31 ppm, and 2.51-2.70 ppm. Preferably, ten or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

In one embodiment, the invention utilizes fifteen or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, 3.17-3.31 ppm, and 2.51-2.70 ppm. Preferably, fifteen or more chemical shift region(s) selected from: 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

In a particularly preferred embodiment, the invention utilizes all of the following chemical shift region(s): 0.86-0.92 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, 3.17-3.31 ppm, and 2.51-2.70 ppm.

In some embodiments, chemical shift ranges encompassing a plurality of the narrower ranges provided above are employed, for example one or more of the following chemical shift region(s) may be employed 0.80-0.92 ppm, 1.15-1.39 ppm, 1.53-1.61 ppm, 1.88-2.10 ppm, 2.20-2.49 ppm, 2.51-2.70 ppm, 3.17-3.95 ppm, 4.63-4.66 ppm, and 5.22-5.38 ppm

The terms “subject” and “patient” are used synonymously herein. The “subject” may be a mammal, and preferably the subject is a human subject. A subject may be a subject that has or, preferably, is suspected of having, cancer. In some embodiments, e.g. when it is desired to diagnose whether a subject has a primary or secondary cancer, a subject may be a subject that has cancer.

Preferably, a subject is a subject that has presented with non-specific symptoms/signs. Such non-specific symptoms/signs may include unexplained weight loss, severe unexplained fatigue, persistent nausea or appetite loss, new atypical pain, unexplained laboratory test finding, and/or GP clinical suspicion of cancer or serious disease (GP ‘gut feeling’).

A subject may be a subject that is at least 35 years old, preferably at least 40 years old.

The sample that is to be tested using the method of the invention may be derived from any suitable biofluid. Thus, the sample is preferably a biofluid sample. In one embodiment, the biofluid is selected from blood, cerebrospinal fluid (CSF), or urine that has been obtained from a subject. Preferably the sample is a blood sample.

The term blood comprises whole blood, blood serum (henceforth “serum”) and blood plasma (henceforth “plasma”), preferably serum. Serum and plasma are derived from blood and thus may be considered as specific subtypes within the broader genus “blood”. Processes for obtaining serum or plasma from blood are known in the art. For example, it is known in the art that blood can be subjected to centrifugation in order to separate red blood cells, white blood cells, and plasma. Serum is defined as plasma that lacks clotting factors. Serum can be obtained by centrifugation of blood in which the clotting process has been triggered. Optionally, this can be carried out in specialised centrifuge tubes designed for this purpose.

Thus, in a particularly preferred embodiment, a metabolite referred to herein is a blood serum metabolite.

A sample for use in a method of the present invention can be derived from a biofluid that has undergone processing after being obtained from a test subject. Alternatively, a sample can be derived from a biofluid that has not undergone any processing after being obtained from a test subject.

The methods of the invention may use samples that have undergone minimal or zero processing before testing. This provides a significant advantage over prior art methods in terms of time, cost and practicality. By way of example, a blood sample obtained from a test subject may be tested directly using the method of the present invention, without further processing. Serum and plasma samples can be readily obtained from blood samples using simple and readily available techniques that are well known in the art, as described above.

A sample for use in a method of the invention may be a cell-free sample. In other words, the sample of the invention may be processed to remove cells. The term “cell-free samples” are samples that contain substantially no cells. The term “substantially no” when used in the context of cells herein may mean less than 10,000, 5,000, 1,000, 100 or 10 cells/ml. The term “substantially no” when used in the context of cells herein preferably means less than 1,000 cells/ml, more preferably no cells. In some embodiments, the term “substantially no” when used in the context of cells herein may be expressed in absolute amounts. For example, the term “substantially no” when used in the context of cells herein may mean less than 10,000, 5,000, 1,000, 100 or 10 cells. Preferably less than 1,000 cells, more preferably no cells.

The methods of the invention comprise comparing a concentration of a metabolite to a reference standard. Similarly, the methods of the invention may comprise comparing an intensity of one or more chemical shift regions of a ¹H-NMR spectrum of a sample with a reference standard.

In one embodiment, a reference standard comprises (or consists of) a sample (e.g. a biofluid sample described herein) obtained from a reference subject or subjects, wherein the reference subject is a subject other than the subject being tested in a method of the invention.

In one embodiment, a “reference standard” comprises (or consists of) a set of data relating to the concentration of one or more metabolites, and/or the intensity of one or more chemical shift regions of a ¹H-NMR spectrum, obtained from a reference subject or subjects, wherein the reference subject is a subject other than the subject being tested in a method of the invention. The set of data may be derived by measuring the concentration of said one or more metabolites and/or measuring the intensity of one or more chemical shift regions of a ¹H-NMR spectrum. Said measuring may be carried out using any suitable technique known in the art or described herein. It is particularly preferred that the set of data corresponding to the reference sample are obtained (or have been obtained) using the same or a similar technique used to obtain the concentration of the one or more metabolites or one or more chemical shift regions (respectively) in the sample being tested. As part of his common general knowledge, the skilled person knows which variables in an experimental protocol can be varied without affecting comparability of data and those that cannot be varied, and will thus select an appropriate experimental protocol to ensure comparability between a sample from a subject and a reference standard. Most preferably, the same technique and protocol will be used to obtain the concentration of the one or more metabolites or one or more chemical shift regions (respectively) in the sample and in the reference standard.

In some embodiments, a reference standard may be a dataset constructed based on a knowledge of metabolite concentrations, and/or chemical shift intensities, that are indicative of the presence of cancer, the absence of cancer, the presence of a primary cancer or the presence of a secondary cancer. In some embodiments, a reference standard may be constructed based on metabolite concentrations and/or chemical shift intensities for a known cancer and/or non-cancer population. In other words, in some embodiments, a reference standard does not correspond to an actual sample obtained from a reference subject. However, it is preferred that a reference standard comprises (or consists of) a set of data relating to the concentration of one or more metabolites, and/or the intensity of one or more chemical shift regions of a ¹H-NMR spectrum, obtained from a reference subject or subjects, wherein the reference subject is a subject other than the subject being tested in a method of the invention.

In one embodiment, the reference standard comprises (or consists of) a set of data relating to the concentration of said one or more metabolites, and/or the intensity of one or more chemical shift regions of a ¹H-NMR spectrum, in a sample or samples derived from a single reference subject. In other embodiments, the reference standard comprises (or consists of) a set of data relating to the concentration of said one or more metabolites, and/or the intensity of one or more chemical shift regions of a ¹H-NMR spectrum, in a sample or samples derived from a plurality of reference subjects (e.g. two or more reference subjects). Thus, in one embodiment, the reference standard is derived by pooling data obtained from two or more (e.g. three, four, five, 10, 15, 20 or 25) reference subjects and calculating an average (for example, mean or median) concentration for each metabolite, and/or an average intensity of one or more chemical shift regions of a ¹H-NMR spectrum. Thus, the reference standard may reflect average concentrations of said one or more metabolites, and/or average intensities of chemical shift regions of a ¹H-NMR spectrum, in a given population of reference subjects. Said concentrations and/or intensities may be expressed in absolute or relative terms, in the same manner as described above in relation to the sample that is to be tested using the method of the invention.

In one embodiment, a method of the invention comprises the use of a plurality of reference standards. In such embodiments, a method may comprise the use of a non-cancer reference standard and a cancer reference standard or the use of primary cancer reference standard and a secondary cancer reference standard. The use of multiple reference standards is particularly preferred when it is necessary to diagnose not only whether or not a subject has cancer, but also whether a subject has a primary or secondary cancer.

In some embodiments, the methods of the present invention comprise comparing measured concentrations of metabolites to the concentration of said metabolites (respectively) in both a cancer and a non-cancer reference standard (or a plurality of cancer and non-cancer reference standards) and determining to which reference standard the sample is most similar (thus allowing a determination/diagnosis according to a method of the invention).

In some embodiments, the methods of the present invention comprise comparing measured intensities of chemical shift regions of a ¹H-NMR spectrum to the intensity of said chemical shift regions of a ¹H-NMR spectrum (respectively) in both a cancer and a non-cancer reference standard (or a plurality of cancer and non-cancer reference standards) and determining to which reference standard the sample is most similar (thus allowing a determination/diagnosis according to a method of the invention).

A metabolite concentration in a reference standard may have been obtained (e.g. quantified) prior to carrying out a method of the invention.

When comparing concentrations between the sample and the reference standard, the way in which the concentrations are expressed is matched between the sample and the reference standard. Thus, an absolute concentration can be compared with an absolute concentration, and a relative concentration can be compared with a relative concentration.

An intensity of a chemical shift region of a ¹H-NMR spectrum in a reference standard may have been obtained (e.g. quantified) prior to carrying out a method of the invention.

When comparing intensities between the sample and the reference standard, the way in which the intensities are expressed is matched between the sample and the reference standard. Thus, an absolute intensity can be compared with an absolute intensity, and a relative intensity can be compared with a relative concentration. Moreover, the ¹H-NMR protocol used for obtaining a spectrum for the sample and reference standard should preferably be the same.

The reference standard is preferably derived from the same sample type (e.g. biofluid) as the sample that is being tested, thus allowing for an appropriate comparison between the metabolites and/or chemical shifts.

The methods of the present invention are in vitro methods. Thus, the methods can be carried out in vitro on an isolated sample that has been obtained from a subject.

The methods of the invention may comprise comparing the (measured) concentrations of one or more metabolites to make a diagnosis. Thus, said (measured) concentrations may correlate with the presence of cancer (e.g. primary or secondary cancer). Said diagnosis may be based on measuring/identifying a concentration difference. The term “concentration difference” embraces both positive and negative differences. Thus, a concentration difference can mean that the concentration of a metabolite is higher in the sample being tested than in the reference standard. Alternatively, a concentration difference can mean that the concentration of a metabolite is lower in the sample than in the reference standard.

Similarly, methods of the invention may comprise comparing the (measured) intensities of one or more chemical shift regions of a ¹H-NMR spectrum to make a diagnosis. Thus, said (measured) intensities may correlate with the presence of cancer (e.g. primary or secondary cancer). Said diagnosis may be based on measuring/identifying a difference in intensity. The term “difference in intensity” embraces both positive and negative differences. Thus, a difference in intensity can mean that the intensity of a chemical shift region is higher in the sample being tested than in the reference standard. Alternatively, a difference in intensity can mean that the intensity of a chemical shift region is lower in the sample than in the reference standard.

The comparison and/or identification of the presence or absence of a concentration difference (as described above) can be achieved using methods of statistical analysis. The comparison and/or identification of the presence or absence of a difference in intensity of a chemical shift region (as described above) can be achieved using methods of statistical analysis. In one embodiment, a method of statistical analysis suitable for use in the present invention includes orthogonal partial least squares discriminate analysis (OPLS-DA).

Identifying a higher or lower concentration of a metabolite or intensity of a chemical shift region relative to the same metabolite or chemical shift region (respectively) in/of a reference standard preferably means identifying a statistically significant higher or lower concentration or intensity. Identifying the same concentration of a metabolite or intensity of a chemical shift region relative to the same metabolite or chemical shift region (respectively) in/of a reference standard preferably means identifying no statistically significant concentration difference or difference in intensity (respectively). More preferably, identifying the same concentration of a metabolite or intensity of a chemical shift region relative to the same metabolite or chemical shift region (respectively) in/of a reference standard preferably means identifying no concentration difference or difference in intensity (respectively).

It is particularly preferred that when carrying out the methods for diagnosing cancer that at least one reference standard is a non-cancer reference standard. The term “non-cancer reference standard” is a reference standard that is representative of a subject that does not have cancer. Preferably, a “non-cancer reference standard” is representative of a healthy subject that does not have any diseases. Thus, a “non-cancer reference standard” may be a reference standard that has been obtained from a subject that does not have (and has not had) cancer (or that did not have cancer when the reference standard was obtained). Preferably, a “non-cancer reference standard” may be a reference standard that has been obtained from a healthy subject.

Preferably, a non-cancer reference standard may be a reference standard that is representative of a subject that does not have (and preferably has not had) a tumour (or that did not have a tumour when the reference standard was obtained). For example, a non-cancer reference standard may be a reference standard that is representative of a subject that does not have (and preferably has not had) a benign, premalignant or malignant tumour (or that did not have a tumour when the reference standard was obtained).

In one embodiment, a non-cancer reference standard is a reference standard that is representative of a subject that does not have (and has not had) cancer but has one or more symptoms of ill health. In a most preferred embodiment, a non-cancer reference standard may be a reference standard that is representative of a subject that does not have (and has not had) cancer but has one or more non-specific signs (e.g. non-specific symptoms). For example, the one or more non-specific signs may be one or more of the following (non-specific signs): unexplained weight loss, severe unexplained fatigue, persistent nausea or appetite loss, new atypical pain, an unexplained laboratory test finding, and/or GP clinical suspicion of cancer or serious disease (GP ‘gut feeling’). (See Nicholson et al; BMJ Open 2018; DOI: 10.1136/bmjopen-2017-018168, the contents of which is incorporated herein by reference). Advantageously, the use of such a reference standard allows a clinician to determine whether a subject presenting with one or more symptoms of ill health (preferably one or more non-specific signs) is a subject that is unwell but that does not have cancer (e.g. has an alternative disorder) or is a subject that is unwell and has cancer.

A non-cancer reference standard may additionally be representative of a subject that is at least 35 years old, preferably at least 40 years old.

In one embodiment, a cancer is diagnosed when the concentration of one or more metabolite(s) selected from: glucose, β-hydroxybutyrate, β-hydroxybutyric acid, NAC, threonine, and myo-inositol is higher in the sample relative to the reference standard, wherein the reference standard is a non-cancer reference standard.

In one embodiment, a cancer is diagnosed when the concentration of one or more metabolite(s) selected from: a lipoprotein, lactate, lactic acid, acetate, and acetic acid is lower in the sample relative to the reference standard, wherein the reference standard is a non-cancer reference standard.

Preferably, a cancer is diagnosed when the concentration of one or more metabolite(s) selected from: glucose, β-hydroxybutyrate, β-hydroxybutyric acid, NAC, threonine, and myo-inositol is higher in the sample relative to the reference standard, wherein the reference standard is a non-cancer reference standard; and when the concentration of one or more metabolite(s) selected from: a lipoprotein, lactate, lactic acid, acetate, and acetic acid is lower in the sample relative to the reference standard, wherein the reference standard is a non-cancer reference standard.

Advantageously, when the reference standard is a non-cancer reference standard, one or more of the following metabolite(s) find particular utility in diagnosing either a primary cancer or a secondary cancer: a lipoprotein (preferably HDL), lactate, lactic acid, NAC, threonine, glucose, myo-inositol, and/or free choline.

Advantageously, when the reference standard is a non-cancer reference standard, one or more of the following metabolite(s) find particular utility in diagnosing a secondary cancer: a lipoprotein (preferably VLDL), β-hydroxybutyrate, β-hydroxybutyric acid, acetate, acetic acid, citrate, and/or citric acid. Preferably, one or metabolite(s) selected from: a lipoprotein (preferably VLDL), β-hydroxybutyrate, β-hydroxybutyric acid, acetate, and acetic acid.

In one embodiment, a cancer is not diagnosed when the concentration of one or more metabolite(s) selected from: glucose, β-hydroxybutyrate, β-hydroxybutyric acid, NAC, threonine, and myo-inositol is the same or lower in the sample relative to the reference standard, wherein the reference standard is a non-cancer reference standard.

In one embodiment, a cancer is not diagnosed when the concentration of one or more metabolite(s) selected from: a lipoprotein, lactate, lactic acid, acetate, and acetic acid is the same or higher in the sample relative to the reference standard, wherein the reference standard is a non-cancer reference standard.

Preferably, a cancer is not diagnosed when the concentration of one or more metabolite(s) selected from: glucose, β-hydroxybutyrate, β-hydroxybutyric acid, NAC, threonine, and myo-inositol is the same or lower in the sample relative to the reference standard, wherein the reference standard is a non-cancer reference standard; and when the concentration of one or more metabolite(s) selected from: a lipoprotein, lactate, lactic acid, acetate, and acetic acid is the same or higher in the sample relative to the reference standard, wherein the reference standard is a non-cancer reference standard.

In one embodiment, cancer is diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is higher in the sample relative to the reference standard, wherein the reference standard is a non-cancer reference standard.

In one embodiment, cancer is diagnosed when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is lower in the sample relative to the reference standard, wherein the reference standard is a non-cancer reference standard.

Preferably, cancer is diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is higher in the sample relative to the reference standard, wherein the reference standard is a non-cancer reference standard; and when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is lower in the sample relative to the reference standard, wherein the reference standard is a non-cancer reference standard.

Advantageously, when the reference standard is a non-cancer reference standard, one or more of the following chemical shift region(s) find particular utility in diagnosing a secondary cancer: 0.86-0.92 ppm, 1.19-1.21 ppm, 2.27-2.45 ppm, 1.30-1.39 ppm, 1.53-1.61 ppm, 1.88-1.93 ppm, 2.20-2.26 ppm, and/or 2.51-2.70 ppm. Preferably, one or chemical shift region(s) selected from: 0.86-0.92 ppm, 1.19-1.21 ppm, 2.27-2.45 ppm, 1.30-1.39 ppm, 1.53-1.61 ppm, 1.88-1.93 ppm, and 2.20-2.26 ppm.

In one embodiment, cancer is not diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is the same or lower in the sample relative to the reference standard, wherein the reference standard is a non-cancer reference standard.

In one embodiment, cancer is not diagnosed when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is the same or higher in the sample relative to the reference standard, wherein the reference standard is a non-cancer reference standard.

Preferably, cancer is not diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is the same or lower in the sample relative to the reference standard, wherein the reference standard is a non-cancer reference standard; and when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is the same or higher in the sample relative to the reference standard, wherein the reference standard is a non-cancer reference standard.

In one embodiment, at least one cancer reference standard may be used in a method of the invention. The term “cancer reference standard” is a reference standard that is representative of a subject that has cancer. In one embodiment, a “cancer reference standard” is representative of a subject that has a primary cancer, i.e. it is a “primary cancer reference standard”. In one embodiment, a “cancer reference standard” is representative of a subject that has a secondary cancer, i.e. it is a “secondary cancer reference standard”. Thus, a “cancer reference standard” may be a reference standard that has been obtained from a subject that has a primary cancer (or had a primary cancer at the time the reference standard was obtained). Alternatively, a “cancer reference standard” may be a reference standard that has been obtained from a subject that has a secondary cancer (or had a secondary cancer at the time the reference standard was obtained). Such a subject preferably also has a primary cancer.

In one embodiment, a cancer reference standard is a reference standard that is representative of a subject that has cancer or has had cancer (preferably has cancer) and has one or more symptoms of ill health. Preferably, a cancer reference standard may be a reference standard that is representative of a subject that has or has had cancer (preferably has cancer) cancer and has one or more non-specific signs (e.g. non-specific symptoms). For example, the one or more non-specific signs may be one or more of the following (non-specific signs): unexplained weight loss, severe unexplained fatigue, persistent nausea or appetite loss, new atypical pain, an unexplained laboratory test finding, and/or GP clinical suspicion of cancer or serious disease (GP ‘gut feeling’). (See Nicholson et al; BMJ Open 2018; DOI: 10.1136/bmjopen-2017-018168, the contents of which is incorporated herein by reference).

A cancer reference standard may additionally be representative of a subject that is at least years old, preferably at least 40 years old.

In one embodiment, cancer is diagnosed when the concentration of one or more metabolite(s) selected from: glucose, β-hydroxybutyrate, β-hydroxybutyric acid, NAC, threonine, and myo-inositol is the same or higher in the sample relative to the reference standard, wherein the reference standard is a cancer reference standard.

In one embodiment, a cancer is diagnosed when the concentration of one or more metabolite(s) selected from: a lipoprotein, lactate, lactic acid, acetate, and acetic acid is the same or lower in the sample relative to the reference standard, wherein the reference standard is a cancer reference standard.

Preferably, cancer is diagnosed when the concentration of one or more metabolite(s) selected from: glucose, β-hydroxybutyrate, β-hydroxybutyric acid, NAC, threonine, and myo-inositol is the same or higher in the sample relative to the reference standard, wherein the reference standard is a cancer reference standard; and when the concentration of one or more metabolite(s) selected from: a lipoprotein, lactate, lactic acid, acetate, and acetic acid is the same or lower in the sample relative to the reference standard, wherein the reference standard is a cancer reference standard.

In one embodiment, cancer is not diagnosed when the concentration of one or more metabolite(s) selected from: glucose, β-hydroxybutyrate, β-hydroxybutyric acid, NAC, threonine, and myo-inositol is lower in the sample relative to the reference standard, wherein the reference standard is a cancer reference standard.

In one embodiment, a cancer is not diagnosed when the concentration of one or more metabolite(s) selected from: a lipoprotein, lactate, lactic acid, acetate, and acetic acid is higher in the sample relative to the reference standard, wherein the reference standard is a cancer reference standard.

Preferably, cancer is not diagnosed when the concentration of one or more metabolite(s) selected from: glucose, β-hydroxybutyrate, β-hydroxybutyric acid, NAC, threonine, and myo-inositol is lower in the sample relative to the reference standard, wherein the reference standard is a cancer reference standard; and when the concentration of one or more metabolite(s) selected from: a lipoprotein, lactate, lactic acid, acetate, and acetic acid is higher in the sample relative to the reference standard, wherein the reference standard is a cancer reference standard.

In one embodiment, cancer is not diagnosed when the concentration of one or more metabolite(s) selected from: glucose, NAC, threonine, and myo-inositol is lower in the sample relative to the reference standard, wherein the reference standard is a secondary cancer reference standard.

In one embodiment, cancer is not diagnosed when the concentration of β-hydroxybutyrate and/or β-hydroxybutyric acid is lower in the sample relative to the reference standard, wherein the reference standard is a primary cancer reference standard.

In one embodiment, a cancer is not diagnosed when the concentration of one or more metabolite(s) selected from: a lipoprotein, lactate, lactic acid, acetate, and acetic acid is higher in the sample relative to the reference standard, wherein the reference standard is a secondary cancer reference standard.

Preferably, a cancer is not diagnosed when the concentration of one or more metabolite(s) selected from: glucose, NAC, threonine, and myo-inositol is lower in the sample relative to the reference standard, wherein the reference standard is a secondary cancer reference standard; and when the concentration of one or more metabolite(s) selected from: a lipoprotein, lactate, lactic acid, acetate, and acetic acid is higher in the sample relative to the reference standard, wherein the reference standard is a secondary cancer reference standard; and optionally when the concentration of β-hydroxybutyrate and/or β-hydroxybutyric acid is lower in the sample relative to the reference standard, wherein the reference standard is a primary cancer reference standard.

In one embodiment, cancer is diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is the same or higher in the sample relative to the reference standard, wherein the reference standard is a cancer reference standard.

In one embodiment, cancer is diagnosed when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is the same or lower in the sample relative to the reference standard, wherein the reference standard is a cancer reference standard.

Preferably, cancer is diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is the same or higher in the sample relative to the reference standard, wherein the reference standard is a cancer reference standard; and when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is the same or lower in the sample relative to the reference standard, wherein the reference standard is a cancer reference standard.

In one embodiment, cancer is not diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is lower in the sample relative to the reference standard, wherein the reference standard is a cancer reference standard.

In one embodiment, cancer is not diagnosed when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is higher in the sample relative to the reference standard, wherein the reference standard is a cancer reference standard.

Preferably, cancer is not diagnosed when the intensity of one or more chemical shift region(s) selected from: 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is lower in the sample relative to the reference standard, wherein the reference standard is a cancer reference standard; and when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is higher in the sample relative to the reference standard, wherein the reference standard is a cancer reference standard.

In one embodiment, cancer is not diagnosed when the intensity of one or more chemical shift region(s) selected from: 3.17-3.95 ppm, 4.63-4.66 ppm, 5.22-5.25 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is lower in the sample relative to the reference standard, wherein the reference standard is a secondary cancer reference standard.

In one embodiment, cancer is not diagnosed when the intensity of one or more chemical shift region(s) selected from: 1.19-1.21 ppm and 2.27-2.45 ppm is lower in the sample relative to the reference standard, wherein the reference standard is a primary cancer reference standard.

In one embodiment, a cancer is not diagnosed when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is higher in the sample relative to the reference standard, wherein the reference standard is a secondary cancer reference standard.

Preferably, cancer is not diagnosed when the intensity of one or more chemical shift region(s) selected from: 3.17-3.95 ppm, 4.63-4.66 ppm, 5.22-5.25 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is lower in the sample relative to the reference standard, wherein the reference standard is a secondary cancer reference standard; and when the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is higher in the sample relative to the reference standard, wherein the reference standard is a secondary cancer reference standard; and optionally when the intensity of one or more chemical shift region(s) selected from: 1.19-1.21 ppm and 2.27-2.45 ppm is lower in the sample relative to the reference standard, wherein the reference standard is a primary cancer reference standard.

The methods of the invention may further comprise comparing a concentration of citrate and/or citric acid present in a sample obtained from a subject with the concentration of citrate and/or citric acid (respectively) in a reference standard. Cancer may be diagnosed when the concentration of citrate and/or citric acid is higher in the sample relative to the reference standard, wherein the reference standard is a non-cancer reference standard. Cancer may also be diagnosed when the concentration of citrate and/or citric acid is the same or higher in the sample relative to the reference standard, wherein the reference standard is a cancer reference standard. In one embodiment, cancer is not diagnosed when the concentration of citrate and/or citric acid is the same or lower in the sample relative to the reference standard, wherein the reference standard is a non-cancer reference standard. In one embodiment, cancer is not diagnosed when the concentration of citrate and/or citric acid is lower in the sample relative to the reference standard, wherein the reference standard is a secondary cancer reference standard.

The methods of the invention may further comprise comparing an intensity of a chemical shift region of a ¹H-NMR spectrum of a sample obtained from a subject with the intensity of the same one or more chemical shift region of a ¹H-NMR reference standard, wherein the chemical shift region is 2.51-2.70 ppm. Cancer may be diagnosed when the intensity of the chemical shift region of the ¹H-NMR spectrum of the sample obtained from the subject is higher in the sample relative to the reference standard, wherein the reference standard is a non-cancer reference standard. Cancer may be diagnosed when the intensity of the chemical shift region of the ¹H-NMR spectrum of the sample obtained from the subject is the same or higher in the sample relative to the reference standard, wherein the reference standard is a cancer reference standard. In one embodiment, cancer is not diagnosed when the intensity of the chemical shift region of the ¹H-NMR spectrum of the sample obtained from the subject is the same or lower in the sample relative to the reference standard, wherein the reference standard is a non-cancer reference standard. In one embodiment, cancer is not diagnosed when the intensity of the chemical shift region of the ¹H-NMR spectrum of the sample obtained from the subject is lower in the sample relative to the reference standard, wherein the reference standard is a secondary cancer reference standard.

The methods may further comprise comparing a concentration of lipoprotein and/or free choline in a sample obtained from a subject with the concentration of lipoprotein and/or free choline (respectively) in a reference standard. The metabolite lipoprotein and/or free choline may provide an indication of whether a cancer so diagnosed is a primary or secondary cancer. For example, a secondary cancer may be diagnosed when the concentration of lipoprotein and/or free choline is higher in the sample relative to the reference standard, wherein the reference standard is a non-cancer reference standard. In one embodiment, a secondary cancer may be diagnosed when the concentration of lipoprotein and/or free choline is the same or higher in the sample relative to the reference standard, wherein the reference standard is a secondary cancer reference standard. Similarly, a primary cancer may be diagnosed when the concentration of lipoprotein and/or free choline is lower in the sample relative to the reference standard, wherein the reference standard is a non-cancer reference standard. In one embodiment, a primary cancer may be diagnosed when the concentration of lipoprotein and/or free choline is the same or lower in the sample relative to the reference standard, wherein the reference standard is a primary cancer reference standard.

The methods may further comprise comparing an intensity of a chemical shift region of a ¹H-NMR spectrum of a sample obtained from a subject with the intensity of the same one or more chemical shift region of a ¹H-NMR reference standard, wherein the chemical shift region is 3.17-3.31 ppm. The chemical shift region of 3.17-3.31 ppm may provide an indication of whether a cancer so diagnosed is a primary or secondary cancer. For example, a secondary cancer may be diagnosed when the intensity of the chemical shift region of the ¹H-NMR spectrum of the sample obtained from the subject is higher in the sample relative to the reference standard, wherein the reference standard is a non-cancer reference standard. In one embodiment, a secondary cancer is diagnosed when the intensity of the chemical shift region of the ¹H-NMR spectrum of the sample obtained from the subject is the same or higher in the sample relative to the reference standard, wherein the reference standard is a secondary cancer reference standard. Similarly, a primary cancer may be diagnosed when the intensity of the chemical shift region of the ¹H-NMR spectrum of the sample obtained from the subject is lower in the sample relative to the reference standard, wherein the reference standard is a non-cancer reference standard. In one embodiment, a primary cancer is diagnosed when the intensity of the chemical shift region of the ¹H-NMR spectrum of the sample obtained from the subject is the same or lower in the sample relative to the reference standard, wherein the reference standard is a primary cancer reference standard.

In one embodiment, a secondary cancer is diagnosed when the concentration of one or more metabolite(s) selected from: a lipoprotein, β-hydroxybutyrate, β-hydroxybutyric acid, lactate, lactic acid, acetate, acetic acid, and free choline is higher in the sample relative to the reference standard, wherein the reference standard is a primary cancer reference standard.

In one embodiment, a secondary cancer is diagnosed when the concentration of one or more metabolite(s) selected from: NAC, threonine, myo-inositol, and glucose is lower in the sample relative to the reference standard, wherein the reference standard is a primary cancer reference standard.

Preferably, a secondary cancer is diagnosed when the concentration of one or more metabolite(s) selected from: a lipoprotein, β-hydroxybutyrate, β-hydroxybutyric acid, lactate, lactic acid, acetate, acetic acid, and free choline is higher in the sample relative to the reference standard, wherein the reference standard is a primary cancer reference standard; and when the concentration of one or more metabolite(s) selected from: NAC, threonine, myo-inositol, and glucose is lower in the sample relative to the reference standard, wherein the reference standard is a primary cancer reference standard.

In one embodiment, a secondary cancer is diagnosed when the concentration of one or more metabolite(s) selected from: a lipoprotein, β-hydroxybutyrate, β-hydroxybutyric acid, lactate, lactic acid, acetate, acetic acid, and free choline is the same or higher in the sample relative to the reference standard, wherein the reference standard is a secondary cancer reference standard.

In one embodiment, a secondary cancer is diagnosed when the concentration of one or more metabolite(s) selected from: NAC, threonine, myo-inositol, and glucose is the same or lower in the sample relative to the reference standard, wherein the reference standard is a secondary cancer.

Preferably, a secondary cancer is diagnosed when the concentration of one or more metabolite(s) selected from: a lipoprotein, β-hydroxybutyrate, β-hydroxybutyric acid, lactate, lactic acid, acetate, acetic acid, and free choline is the same or higher in the sample relative to the reference standard, wherein the reference standard is a secondary cancer reference standard; and when the concentration of one or more metabolite(s) selected from: NAC, threonine, myo-inositol, and glucose is the same or lower in the sample relative to the reference standard, wherein the reference standard is a secondary cancer.

In one embodiment, a primary cancer is diagnosed when the concentration of one or more metabolite(s) selected from: a lipoprotein, β-hydroxybutyrate, β-hydroxybutyric acid, lactate, lactic acid, acetate, acetic acid, and free choline is the same or lower in the sample relative to the reference standard, wherein the reference standard is a primary cancer reference standard.

In one embodiment, a primary cancer is diagnosed when the concentration of one or more metabolite(s) selected from: NAC, threonine, myo-inositol, and glucose is the same or higher in the sample relative to the reference standard, wherein the reference standard is primary cancer reference standard.

Preferably, a primary cancer is diagnosed when the concentration of one or more metabolite(s) selected from: a lipoprotein, β-hydroxybutyrate, β-hydroxybutyric acid, lactate, lactic acid, acetate, acetic acid, and free choline is the same or lower in the sample relative to the reference standard, wherein the reference standard is a primary cancer reference standard; and when the concentration of one or more metabolite(s) selected from: NAC, threonine, myo-inositol, and glucose is the same or higher in the sample relative to the reference standard, wherein the reference standard is primary cancer reference standard.

In one embodiment, a primary cancer is diagnosed when the concentration of one or more metabolite(s) selected from: a lipoprotein, β-hydroxybutyrate, β-hydroxybutyric acid, lactate, lactic acid, acetate, acetic acid, and free choline is lower in the sample relative to the reference standard, wherein the reference standard is a secondary cancer reference standard.

In one embodiment, a primary cancer is diagnosed when the concentration of one or more metabolite(s) selected from: NAC, threonine, myo-inositol, and glucose is higher in the sample relative to the reference standard, wherein the reference standard is a secondary cancer reference standard.

Preferably, a primary cancer is diagnosed when the concentration of one or more metabolite(s) selected from: a lipoprotein, β-hydroxybutyrate, β-hydroxybutyric acid, lactate, lactic acid, acetate, acetic acid, and free choline is lower in the sample relative to the reference standard, wherein the reference standard is a secondary cancer reference standard; and when the concentration of one or more metabolite(s) selected from: NAC, threonine, myo-inositol, and glucose is higher in the sample relative to the reference standard, wherein the reference standard is a secondary cancer reference standard.

In one embodiment, a method may further comprise comparing a concentration of citrate and/or citric acid present in a sample obtained from a subject with the concentration of citrate and/or citric acid (respectively) in a reference standard. A secondary cancer may be diagnosed when the concentration of citrate and/or citric acid is lower in the sample relative to the reference standard, wherein the reference standard is a primary cancer reference standard. In one embodiment, a secondary cancer may be diagnosed when the concentration of citrate and/or citric acid is the same or lower in the sample relative to the reference standard, wherein the reference standard is a secondary cancer. In one embodiment, a primary cancer may be diagnosed when the concentration of citrate and/or citric acid is the same or higher in the sample relative to the reference standard, wherein the reference standard is primary cancer reference standard. In one embodiment, a primary cancer may be diagnosed when the concentration of citrate and/or citric acid is higher in the sample relative to the reference standard, wherein the reference standard is a secondary cancer reference standard.

In one embodiment, a secondary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is higher in the sample relative to the reference standard, wherein the reference standard is a primary cancer reference standard.

In one embodiment, a secondary cancer is diagnosed when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, and 3.17-3.95 ppm, 4.63-4.66 ppm, and 5.22-5.25 ppm is lower in the sample relative to the reference standard, wherein the reference standard is a primary cancer reference standard.

Preferably, a secondary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is higher in the sample relative to the reference standard, wherein the reference standard is a primary cancer reference standard; and when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, and 3.17-3.95 ppm, 4.63-4.66 ppm, and 5.22-5.25 ppm is lower in the sample relative to the reference standard, wherein the reference standard is a primary cancer reference standard.

In one embodiment, a secondary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or higher in the sample relative to the reference standard, wherein the reference standard is a secondary cancer reference standard.

In one embodiment, a secondary cancer is diagnosed when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or lower in the sample relative to the reference standard, wherein the reference standard is a secondary cancer.

Preferably, a secondary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or higher in the sample relative to the reference standard, wherein the reference standard is a secondary cancer reference standard; and when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or lower in the sample relative to the reference standard, wherein the reference standard is a secondary cancer.

In one embodiment, a primary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or lower in the sample relative to the reference standard, wherein the reference standard is a primary cancer reference standard.

In one embodiment, a primary cancer is diagnosed when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or higher in the sample relative to the reference standard, wherein the reference standard is primary cancer reference standard.

Preferably, a primary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or lower in the sample relative to the reference standard, wherein the reference standard is a primary cancer reference standard; and when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or higher in the sample relative to the reference standard, wherein the reference standard is primary cancer reference standard.

In one embodiment, a primary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is lower in the sample relative to the reference standard, wherein the reference standard is a secondary cancer reference standard.

In one embodiment, a primary cancer is diagnosed when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is higher in the sample relative to the reference standard, wherein the reference standard is a secondary cancer reference standard.

Preferably, a primary cancer is diagnosed when the intensity of one or more chemical shift region(s) selected from: 0.86-0.92 ppm, 0.80-0.86 ppm, 1.19-1.21 ppm, 2.27-2.45 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is lower in the sample relative to the reference standard, wherein the reference standard is a secondary cancer reference standard; and when the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is higher in the sample relative to the reference standard, wherein the reference standard is a secondary cancer reference standard.

In one embodiment, a method of the invention may further comprise comparing an intensity of a chemical shift region of a ¹H-NMR spectrum of a sample obtained from a subject with the intensity of the same one or more chemical shift region of a ¹H-NMR reference standard, wherein the chemical shift region is 2.51-2.70 ppm. A secondary cancer may be diagnosed when the intensity of the chemical shift region of the ¹H-NMR spectrum of the sample obtained from the subject is lower in the sample relative to the reference standard, wherein the reference standard is a primary cancer reference standard. A secondary cancer may be diagnosed when the intensity of the chemical shift region of the ¹H-NMR spectrum of the sample obtained from the subject is the same or lower in the sample relative to the reference standard, wherein the reference standard is a secondary cancer. A primary cancer may be diagnosed when the intensity of the chemical shift region of the ¹H-NMR spectrum of the sample obtained from the subject is the same or higher in the sample relative to the reference standard, wherein the reference standard is primary cancer reference standard. A primary cancer may be diagnosed when the intensity of the chemical shift region of the ¹H-NMR spectrum of the sample obtained from the subject is higher in the sample relative to the reference standard, wherein the reference standard is a secondary cancer reference standard.

In one embodiment, a cancer diagnosed by a method of the invention is not an oesophageal cancer and/or a lung cancer.

In one embodiment, the method of the invention further comprises recording the output of at least one step on a data-storage medium. By way of example, the methods of the present invention can generate data relating to the subject, such data being recordable on a data-storage medium (for example, a form of computer memory such as a hard disk, compact disc, floppy disk, or solid state drive). Such data can comprise (or consist of) data relating to the concentration in a sample (from said subject) of any of one or more metabolites (as described herein) and/or data relating to the intensity in a sample (from said subject) of any of one or more chemical shift regions (as described) herein.

In one aspect the invention provides a data-storage medium, comprising data obtained by a method according to the present invention.

In one aspect, the invention provides a computer program product comprising program instructions to cause a processor to perform a method according to the invention.

In another aspect, the invention provides a device for use in a method of the invention, wherein said device is capable of performing the step of identifying: a concentration (e.g. a concentration difference) of one or more metabolites in the sample when compared to the reference standard and/or an intensity (e.g. a difference in intensity) of one or more chemical shift regions of a ¹H-NMR spectrum of a sample obtained from a subject when compared to the reference standard.

In one aspect, the invention provides a method of treating cancer, the method comprising:
(a) obtaining the results of a method according to the invention; and
(b) administering a cancer therapy when a cancer is diagnosed; and
(c) optionally administering a different therapy when the cancer is not diagnosed.

In one aspect, the invention provides a method of treating cancer, the method comprising:
(a) obtaining the results of a method according to the invention; and
(b) administering a primary cancer therapy when a primary cancer is diagnosed; and
(c) optionally administering a different therapy when the primary cancer is not diagnosed.

In one aspect, the invention provides a method of treating cancer, the method comprising:
(a) obtaining the results of a method according to the invention; and
(b) administering a secondary cancer therapy when a secondary cancer is diagnosed; and
(c) optionally administering a different therapy when the secondary cancer is not diagnosed.

Treatment of cancer may be carried out using any cancer therapeutic known in the art. For example, therapy may include surgery, chemotherapy, antibody therapy, radiation therapy, cell therapy (e.g. a bone marrow transplant and/or T-cell therapy), immunotherapy, hormone therapy, targeted drug therapy, cryoabalation, radiofrequency ablation, thermal ablation or combinations thereof. A suitable radiation therapy may be one that uses high-energy photons (e.g. X-rays and/or gamma rays) and/or one that uses high energy particles (e.g. electrons and/or protons).

The term “disorder” as used herein also encompasses a “disease”. In one embodiment, the disorder is a disease. The disorder treated in accordance with the invention is cancer. In one embodiment, the cancer is a primary cancer and/or a secondary cancer.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment.

The term “treat” or “treating” as used herein refers to the disorder and/or a symptom thereof.

Therefore, a therapeutic may be administered to a subject in a therapeutically effective amount or a prophylactically effective amount.

A “therapeutically effective amount” is any amount of a therapeutic formulation, which when administered alone or in combination to a subject for treating said disorder (or a symptom thereof) is sufficient to effect such treatment of the disorder, or symptom thereof.

A “prophylactically effective amount” is any amount of a therapeutic formulation that, when administered alone or in combination to a subject inhibits or delays the onset or reoccurrence of a disorder (or a symptom thereof). In some embodiments, the prophylactically effective amount prevents the onset or reoccurrence of a disorder entirely. “Inhibiting” the onset means either lessening the likelihood of a disorder's onset (or symptom thereof), or preventing the onset entirely.

Administration may be by any route known in the art and will typically be dependent on the nature of the therapeutic to be administered. For example, a therapeutic may be administered orally or parenterally. Methods of parenteral delivery include topical, intra-arterial, intramuscular, subcutaneous, intramedullary, intrathecal, intra-ventricular, intravenous, intraperitoneal, or intranasal administration.

Embodiments related to the various methods of the invention are intended to be applied equally to other methods, therapeutic uses or methods, the data storage medium or device, the computer program product, and vice versa.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. The headings provided herein are not limitations of the various aspects or embodiments of this disclosure.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a metabolite” includes a plurality of such candidate agents and reference to “the metabolites” includes reference to one or more metabolites and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1. Cancer versus non-cancer. (A) Representative OPLS-DA scores plot illustrating separation between confirmed cancer (triangle) and confirmed non-cancer (circle) serum samples. The accuracy (B), sensitivity (C), specificity (D), R² (E), and cumulative Q² (F) of the ensemble of 1000 cancer versus non-cancer models, as determined by classification of an independent test set, is significantly greater than that of random data confirming that the models are well-validated and significant. Kolmogorov-Smirnov test p-values<0.001 are represented by ***.

FIG. 2. Metastatic cancer versus non-cancer. (A) Representative OPLS-DA scores plot illustrating separation between confirmed metastatic cancer (square) and confirmed non-cancer (circle) serum samples. The accuracy (B), sensitivity (C), specificity (D), R² (E), and cumulative Q² (F) of the ensemble of 1000 metastatic cancer versus non-cancer models, as determined by classification of an independent test set, is significantly greater than that of random data confirming that the models are well-validated and significant. Kolmogorov-Smirnov test p-values<0.001 are represented by ***.

FIG. 3. Metastatic cancer versus primary cancer. (A) Representative OPLS-DA scores plot illustrating separation between confirmed metastatic cancer (square) and confirmed primary cancer only (diamond) serum samples. The accuracy (B), sensitivity (C), specificity (D), R² (E), and cumulative Q² (F) of the ensemble of 1000 metastatic cancer versus primary cancer models, as determined by classification of an independent test set, is significantly greater than that of random data confirming that the models are well-validated and significant. Kolmogorov-Smirnov test p-values<0.001 are represented by ***; p<0.01 represented by **.

FIG. 4. Model Validation. (A) OPLS-DA plot showing separation of unwell patients with cancer diagnoses (triangles) from unwell patients with non-cancer diagnoses (circles). (B) Sensitivity (dots), specificity (dashes), and F1 score (continuous) for cancer vs unwell with cancer models at all possible thresholds of classification according to Component 1. Vertical dashed line shows optimal classification threshold. (C) OPLS-DA plot showing separation of patients with primary-only cancer diagnoses (stars) or metastatic cancer diagnoses (squares). (D) Sensitivity (dots), specificity (dashes), and F1 score (continuous) for the primary vs metastatic cancer model at all possible thresholds of classification according to Component 1. Vertical dashed line shows optimal classification threshold. E: ROC curves for classification between all unwell with cancer vs unwell without cancer diagnoses (continuous line; model in panel A) and primary-only vs metastatic cancer diagnoses (dashed line; model in panel C). Small circles on lines indicate points closest to top-left corner (pointed to by black arrows), corresponding to dashed vertical lines in other panels.

### EXAMPLES

### Example 1

### Patient Recruitment

Patients were recruited from an Oxfordshire-based population (UK). Patients were recruited to the SCAN (Suspected CANcer) pathway if they met three essential referral criteria: (1) There was no other urgent referral pathway suitable for that clinical scenario; (2) the patient was ≥40 years old; (3) the patient had at least one of the following (non-specific signs): unexplained weight loss, severe unexplained fatigue, persistent nausea or appetite loss, new atypical pain, unexplained laboratory test finding, GP clinical suspicion of cancer or serious disease (GP ‘gut feeling’). [Nicholson et al; BMJ Open 2018; DOI: 10.1136/bmjopen-2017-018168].

### Biofluid Sample Collection

Blood was collected immediately prior to computerised tomography (CT) imaging. Blood was collected into lithium-heparin tubes or serum coagulator tubes and left to stand at room temperature before centrifugation at 2,200×g for 10 minutes. Plasma or serum were immediately aliquoted and stored at −80° C. Urine was collected as a mid-stream sample into a clean additive free tube then immediately aliquoted and stored at −80° C.

### NMR Metabolomics Analysis

NMR metabolomics analysis of serum, plasma or urine was carried out as previously described (J Crohns Colitis. 2018 Nov. 15; 12(11):1326-1337., Acta Neuropathol Commun. 2017 Dec. 6; 5(1):95). This is explained in more detail below.

### NMR Sample Preparation

Plasma, serum or urine samples were defrosted at room temperature. 150 μL of the plasma, serum or urine was diluted with 450 μL of 75 mM sodium phosphate buffer prepared in D₂O (pH meter reading of 7.4). Samples were then centrifuged at 16,000×g for 3 minutes to remove any precipitate before transferring to a 5 mm NMR tube.

### NMR Spectroscopy

All NMR spectra were acquired using a 700-MHz Bruker AVIII spectrometer operating at 16.4T equipped with a ¹H (¹³C/¹⁵N) TCI cryoprobe. Sample temperature was stable at 298K. ¹H NMR spectra were acquired using a 1D NOESY presaturation scheme for attenuation of the water resonance with a 2 s presaturation. For serum and plasma samples, in addition to the 1D NOESY presaturation scheme, a spin-echo Carr-Purcell-Meiboom-Gill (CPMG) sequence with a T interval of 400 μs, 80 loops, 32 data collections, an acquisition time of 1.5 s, a relaxation delay of 2 s, and a fixed receiver gain was used to supress broad signals arising from large molecular weight blood components. 1H correlation spectroscopy (COSY, TOCSY) spectra were acquired on at least one sample in each classification to aid in metabolite identification. For quality control, pooled samples were spread throughout the run to monitor technical variation.

### NMR Data Pre-Processing

Resulting free induction decays (FIDs) were zero-filled by a factor of 2 and multiplied by an exponential function corresponding to 0.30 Hz line broadening prior to Fourier transformation. All spectra were phased, baseline corrected (using a 3^{rd} degree polynomial), and chemical shifts referenced to the lactate-CHs doublet resonance at δ=1.33 ppm in Topspin 4.0 (Bruker, Germany). Spectra were visually inspected in Topspin for errors in baseline correction, referencing, spectral distortion, or contamination and then imported into Matlab (Mathworks, MA, USA). For blood plasma and blood serum spectra, the regions between 0.20-4.70 ppm and 5.00-9.60 ppm were divided in to 0.01 ppm width ‘buckets’. For urine spectra, the regions between 0.20-4.70 ppm and 5.00-5.70 and 5.96-9.60 ppm were divided in to 0.01 ppm width ‘buckets’. In all cases, the absolute value of the integral of each spectral bucket was Pareto scaled. Resonances were assigned by reference to literature values [Anal Biochem 325:260-272, J Pharm Biomed Anal 33:1103-1115] and the Human Metabolome Database [Nucleic Acids Res 41:D801-807. doi:10.1093/nar/gks1065, Nucleic Acids Res 37:D603-610. doi:10.1093/nar/gkn810, Nucleic Acids Res 35:D521-526. doi:10.1093/nar/gkl923] and further confirmed by inspection of the 2D spectra, spiking of known compounds, and 1D-TOCSY spectra.

### Statistical Analysis

The bucketed integrals were imported into R (R foundation for statistical computing, Vienna, Austria) [Team RC R: A Language and Environment for Statistical Computing. R Found Stat Comput]. All multivariate analysis was carried out using in house R scripts and the ropls package [J Proteome Res 14:3322-3335]. Principal component analysis (PCA) was used to visualize the degree of separation between the disease classifications and detect potential outliers. Orthogonal partial least squares discriminatory analysis (OPLS-DA) was used to generate diagnostic mathematical models. The quality of classification was assessed using a 10-fold external cross-validation scheme with 1000 repetitions in total (to correct for unequal class sizes). This validation scheme involves multiple iterations of splitting the data into training and testing sets. The training data is used to estimate the model parameters and learn the underlying discriminatory patterns between the groups under consideration, whereas the independent test set is employed to assess the accuracy and generalizability of the trained models in the ensemble. The response of the ensemble of models was quantified by calculating the accuracy, sensitivity, and specificity of each model from the predicted classifications of the external, independent test set (i.e. which is not used in model building). It is important to appreciate that the classifier (OPLS-DA) was blinded to the test set during the process of model training. This validation scheme tends to avoid over-fitting and helps assess the generalizability of the model to previously unseen datasets. For an exhaustive discussion on validation see Arlot and Celisse (2010) [Statistics surveys 4:40-79]. These values were compared with those of a null distribution (obtained from randomly permuting the classifications) using the two-sided Kolmogorov-Smirnov test (significant if p-value 0.05 or less). Discriminators were identified by calculating the variable importance (VIP) score. The fold changes of these variables were further investigated by analysis-of-variance (ANOVA) followed by Tukey's honest significant difference (HSD) post-hoc test. The p-values obtained were then corrected for multiple comparisons using the Bonferroni correction.

### Results

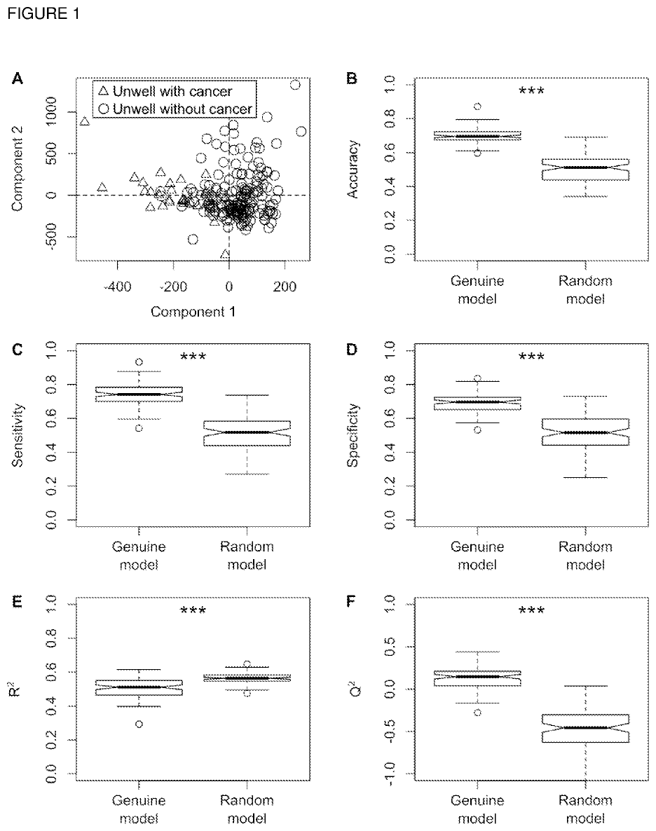

FIG. 1 shows a representative OPLS-DA scores plot illustrating separation between confirmed cancer (triangle) and confirmed non-cancer (circle) serum samples. The results show that the accuracy, sensitivity, specificity, R², and cumulative Q² of the ensemble of 1000 cancer versus non-cancer models, as determined by classification of an independent test set, is significantly greater than that of random data confirming that the models are well-validated and significant. Kolmogorov-Smirnov test p-values<0.001 are represented by ***.

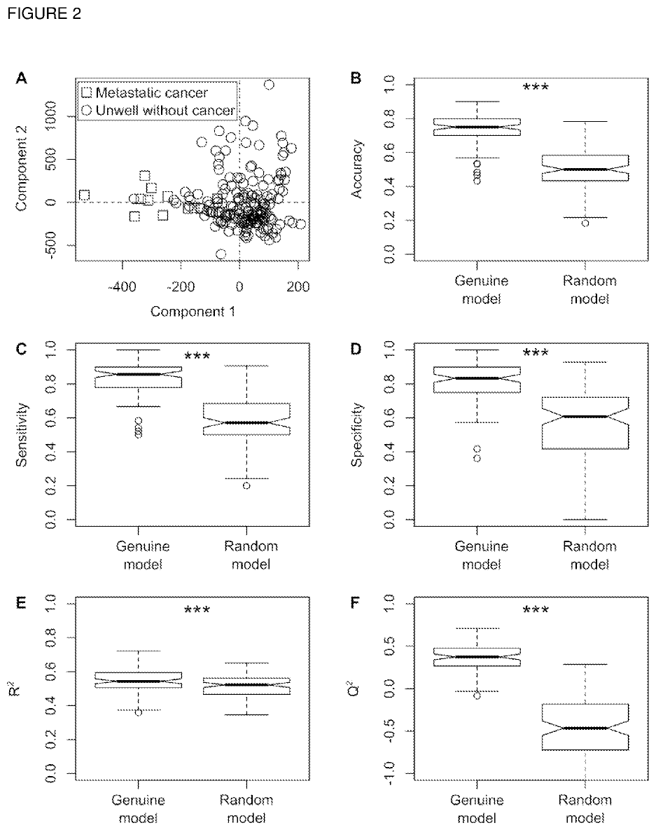

FIG. 2 shows a representative OPLS-DA scores plot illustrating separation between confirmed metastatic cancer (square) and confirmed non-cancer (circle) serum samples. The results show that accuracy, sensitivity, specificity, R², and cumulative Q² of the ensemble of 1000 metastatic cancer versus non-cancer models, as determined by classification of an independent test set, is significantly greater than that of random data confirming that the models are well-validated and significant. Kolmogorov-Smirnov test p-values<0.001 are represented by ***.

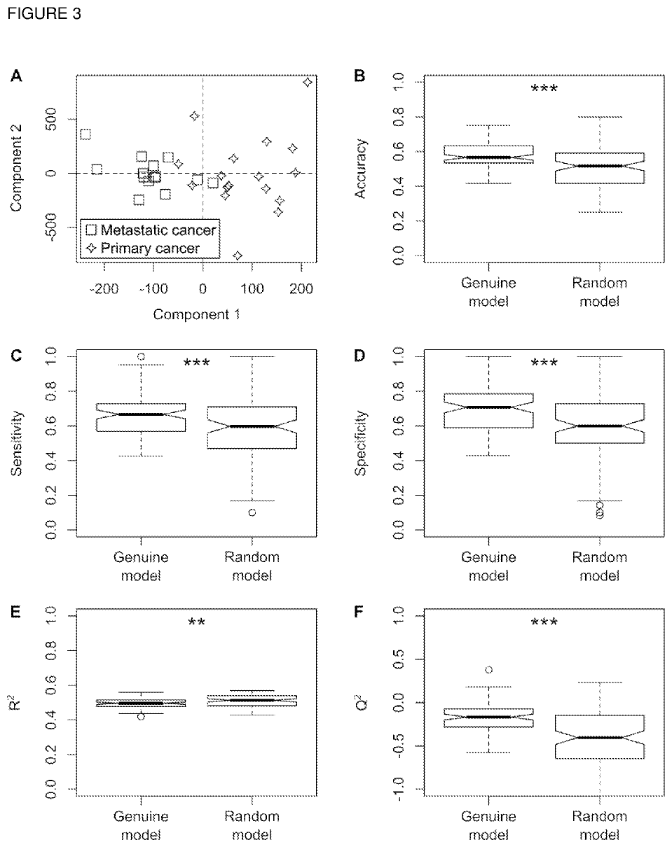

FIG. 3 shows a representative OPLS-DA scores plot illustrating separation between confirmed metastatic cancer (square) and confirmed primary cancer only (diamond) serum samples. The accuracy, sensitivity, specificity, R², and cumulative Q² of the ensemble of 1000 metastatic cancer versus primary cancer models, as determined by classification of an independent test set, is significantly greater than that of random data confirming that the models are well-validated and significant. Kolmogorov-Smirnov test p-values<0.001 are represented by ***; p<0.01 represented by **.

Performance of the various models are shown in Table 1 below, while those regions of the NMR spectra identified as being highly discriminatory are shown in Table 2.

TABLE 1
Summary of the performance of all OPLS-DA models.

| | Cancer | Metastatic cancer | Metastatic cancer |
|---|---|---|---|
| | versus | versus | versus primary |
| | non-cancer | non-cancer | cancer |
| Accuracy | 70 ± 4% | 74 ± 10% | 57 ± 9% |
| Sensitivity | 74 ± 7% | 83 ± 10% | 66 ± 12% |
| Specificity | 69 ± 5% | 82 ± 11% | 66 ± 8% |
| R2 | 0.50 ± 0.06 | 0.53 ± 0.08 | 0.59 ± 0.04 |
| Q2 | 0.13 ± 0.13 | 0.37 ± 0.16 | −0.11 ± 0.17 |

TABLE 2
NMR spectral regions identified as highly
discriminatory by multivariate analysis.
Chemical shift ranges are reported relative to
lactate —CH₃ referenced at 1.33 ppm.
Discriminatory
regions [ppm]
0.80-0.92
1.15-1.39
1.53-1.61
1.88-2.10
2.20-2.49
2.51-2.70
3.17-3.95
4.63-4.66
5.22-5.38

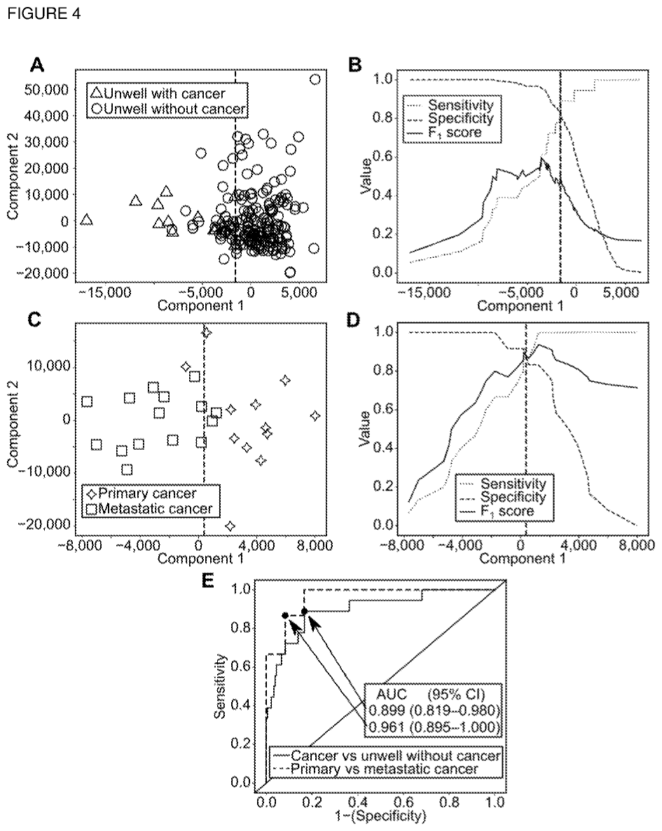

Further characterisation and optimisation of the models improved sensitivity and specificity as confirmed by the analysis presented in FIG. 4.

The statistical analysis and modelling identified a number of metabolites and their concentrations that were shown to be predictive of cancer, as well as metabolites allowing distinction between primary and secondary cancer (see Table 3). Given that the model was trained using confirmed cancer samples from subjects with a wide variety of different cancers, the discriminatory metabolite concentrations shown in Table 3, advantageously, have broad utility in diagnosing any cancer. Similarly, the models were trained with primary and secondary cancers from subjects with a wide variety of different primary and secondary cancers (respectively) and, thus, the discriminatory metabolite concentrations shown in Table 3 have broad utility in differentiating between any primary or secondary cancer.

TABLE 3
Relative concentrations (median) of discriminatory metabolite biomarkers
identified in non-cancer, primary cancer, and metastatic cancer serum samples.
HDL, high density lipoprotein; LDL, low density lipoprotein; VLDL, very low density
lipoprotein; NAC, N-acetylated glycoprotein.

| | Chemical | | | Metastatic |
|---|---|---|---|---|
| | shift range | | Primary | (Secondary) |
| Metabolite | [ppm] | Non-Cancer | Cancer | Cancer |
| Lipoprotein —CH₃ | 0.80-0.86 | 1.29 | 1.06 | 1.16 |
| (HDL/LDL dominated) | | | | |
| Lipoprotein —CH₃ (VLDL | 0.86-0.92 | 14.32 | 12.06 | 13.5 |
| dominated) | | | | |
| β-hydroxybutyrate/β- | 1.19-1.21, | 1.23 | 1.29 | 1.38 |
| hydroxybutyric acid | 2.27-2.45 | | | |
| Lipoprotein —(CH₂)ₙ | 1.15-1.30 | 5.14 | 4.44 | 4.69 |
| (HDL/LDL dominated) | | | | |
| Lactate/lactic acid | 1.31-1.35, | 41.76 | 32.2 | 36.46 |
| | 4.08-4.14 | | | |
| Lipoprotein —(CH₂)ₙ | 1.30-1.39 | 9.95 | 8.07 | 8.94 |
| (VLDL dominated) | | | | |
| Lipoprotein βCH₂ | 1.53-1.61 | 6.51 | 5.1 | 5.85 |
| Acetate/acetic acid | 1.88-1.93 | 1.12 | 0.94 | 0.97 |
| NAC | 1.93-2.10 | 11.5 | 13.79 | 13.04 |
| Lipoprotein αCH₂ | 2.20-2.26 | 3.44 | 2.53 | 3.29 |
| Citrate/citric acid | 2.51-2.70 | 1.26 | 1.43 | 1.35 |
| Lipoprotein —N(CH₃)₃/free | 3.17-3.31 | 3.28 | 2.93 | 3.31 |
| choline | | | | |
| Threonine | 3.57-3.59, | 1.7 | 2.09 | 1.79 |
| | 1.30-1.35, | | | |
| | 4.06-4.3 | | | |
| Myo-inositol | 3.63-3.65, | 2.63 | 3.34 | 2.93 |
| | 3.53-3.58, | | | |
| | 3.93-3.98, | | | |
| | 3.25-3.29 | | | |
| Glucose | 3.17-3.95, | 3.5 | 4.12 | 4.04 |
| | 4.63-4.66, | | | |
| | 5.22-5.25 | | | |

The various metabolites were ranked based on predictive power, as shown in Table 4.

TABLE 4
Rank of the discriminatory metabolite biomarkers
identified in each of the diagnostic multivariate models
where 1 represents the highest ranked variable.

| | Non- | Non- | Primary |
|---|---|---|---|
| | Cancer v. | Cancer v. | Cancer v. |
| | Primary | Metastatic | Metastatic |
| Metabolite | Cancer | Cancer | Cancer |
| Lipoprotein —CH₃ | 4 | 4 | 6 |
| (HDL/LDL dominated) | | | |
| Lipoprotein —CH₃ (VLDL | — | 8 | 1 |
| dominated) | | | |
| β-hydroxybutyrate/β- | — | — | 7 |
| hydroxybutyric acid | | | |
| Lipoprotein —(CH₂)ₙ | 6 | 5 | — |
| (HDL/LDL dominated) | | | |
| Lactate/lactic acid | 8 | 10 | 10 |
| Lipoprotein —(CH₂)ₙ (VLDL | — | 2 | 2 |
| dominated) | | | |
| Lipoprotein βCH₂ | — | 12 | 5 |
| Acetate/acetic acid | — | 14 | — |
| NAC | 2 | 6 | 9 |
| Lipoprotein αCH₂ | — | 11 | 3 |
| Citrate/citric acid | — | 13 | — |
| Lipoprotein —N(CH₃)₃/free | 7 | 3 | — |
| choline | | | |
| Threonine | 5 | 9 | — |
| Myo-inositol | 3 | 7 | 8 |
| Glucose | 1 | 1 | 4 |

In conclusion, a number of metabolites together with their concentrations have been identified that are predictive of cancer. Said metabolites also allow for distinction between primary and secondary cancer. Thus, a diagnosis of cancer can be made by comparing a concentration of a metabolite in a sample from a subject of unknown diagnostic status with one or more of the metabolite concentrations presented in Table 3, above. Similarly, a differentiation can be made between a primary and secondary cancer based on the metabolite concentrations in Table 3.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for diagnosing cancer, the method comprising:
(a) comparing a concentration of one or more metabolite(s) present in a sample obtained from a subject with the concentration of the same one or more metabolite(s) in a reference standard, wherein the one or more metabolite(s) are selected from: a lipoprotein, myo-inositol, glucose, β-hydroxybutyrate, β-hydroxybutyric acid, an N-acetylated glycoprotein (NAC), threonine, lactate, lactic acid, acetate, and acetic acid; and
(b) diagnosing cancer when:
(i) the concentration of one or more metabolite(s) selected from: a lipoprotein, lactate, lactic acid, acetate, and acetic acid is lower in the sample relative to the reference standard, wherein the reference standard is a non-cancer reference standard; and/or
(ii) the concentration of one or more metabolite(s) selected from: myo-inositol glucose, β-hydroxybutyrate, β-hydroxybutyric acid, NAC, and threonine is higher in the sample relative to the reference standard, wherein the reference standard is a non-cancer reference standard; and/or
(iii) the concentration of one or more metabolite(s) selected from: myo-inositol, glucose, β-hydroxybutyrate, β-hydroxybutyric acid, NAC, and threonine is the same or higher in the sample relative to the reference standard, wherein the reference standard is a cancer reference standard; and/or
(iv) the concentration of one or more metabolite(s) selected from: a lipoprotein, lactate, lactic acid, acetate, and acetic acid is the same or lower in the sample relative to the reference standard, wherein the reference standard is a cancer reference standard; or
(c) not diagnosing cancer when:
(i) the concentration of one or more metabolite(s) selected from: myo-inositol, glucose, β-hydroxybutyrate, β-hydroxybutyric acid, NAC, and threonine is the same or lower in the sample relative to the reference standard, wherein the reference standard is a non-cancer reference standard; and/or
(ii) the concentration of one or more metabolite(s) selected from: a lipoprotein, lactate, lactic acid, acetate, and acetic acid is the same or higher in the sample relative to the reference standard, wherein the reference standard is a non-cancer reference standard; and/or
(iii) the concentration of one or more metabolite(s) selected from: myo-inositol, glucose, β-hydroxybutyrate, β-hydroxybutyric acid, NAC, and threonine is lower in the sample relative to the reference standard, wherein the reference standard is a cancer reference standard; and/or
(iv) the concentration of one or more metabolite(s) selected from: a lipoprotein, lactate, lactic acid, acetate, and acetic acid is higher in the sample relative to the reference standard, wherein the reference standard is a cancer reference standard.

2. A method for diagnosing cancer, the method comprising:
(a) comparing a concentration of one or more metabolite(s) present in a sample obtained from a subject with the concentration of the same one or more metabolite(s) in a reference standard, wherein the one or more metabolite(s) are selected from: a lipoprotein, myo-inositol, glucose, β-hydroxybutyrate, β-hydroxybutyric acid, an N-acetylated glycoprotein (NAC), threonine, lactate, lactic acid, acetate, acetic acid, and free choline; or
(b) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 0.80-0.86 ppm, 0.86-0.92 ppm, 1.15-1.30 ppm, 1.30-1.39 ppm, 1.53-1.61 ppm, 2.20-2.26 ppm, 3.17-3.31 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 1.31-1.35 ppm, 4.08-4.14 ppm, and 1.88-1.93 ppm; and
(c) diagnosing cancer or not diagnosing cancer based on the comparison.

3. The method according to claim 1 or 2, wherein the metabolites are at least:
(a) a lipoprotein; and
(b) one or more (e.g. two or more or three or more) metabolite(s) selected from: β-hydroxybutyrate, β-hydroxybutyric acid, threonine, and myo-inositol.

4. The method according to claim 1 or 2, wherein the metabolites are at least:
(a) β-hydroxybutyrate and/or β-hydroxybutyric acid; and
(b) two or more (e.g. three or more, four or more, five or more, six or more or seven or more) metabolites selected from: an N-acetylated glycoprotein (NAC), threonine, myo-inositol, a lipoprotein, lactate, lactic acid, acetate, and acetic acid.

5. The method according to claim 1 or 2, wherein the metabolites are at least:
(a) β-hydroxybutyrate and/or β-hydroxybutyric acid; and
(b) three or more (e.g. four or more, five or more, six or more, seven or more or eight or more) metabolites selected from: glucose, an N-acetylated glycoprotein (NAC), threonine, myo-inositol, a lipoprotein, lactate, lactic acid, acetate, and acetic acid.

6. The method according to claim 1 or 2, wherein the metabolites are at least:
(a) NAC; and
(b) one or more (e.g. two or more, three or more, four or more, five or more, six or more, seven or more, eight or more or nine or more) metabolite(s) selected from: glucose, β-hydroxybutyrate, β-hydroxybutyric acid, threonine, myo-inositol, a lipoprotein, lactate, lactic acid, acetate, and acetic acid.

7. The method according to claim 1 or 2, wherein the biofluid sample is a blood sample (preferably a serum sample) and wherein the one or more metabolite(s) comprise at least myo-inositol.

8. The method according to claim 1 or 2, wherein the sample is a blood serum sample and the one or more metabolite(s) comprise at least a lipoprotein.

9. The method according to claim 1 or 2, wherein the sample is a blood serum sample and the one or more metabolite(s) comprise at least acetate and/or acetic acid.

10. The method according to claim 1 or 2, wherein the sample is a blood serum sample and the one or more (e.g. two or more or three or more) metabolite(s) are selected from: myo-inositol, a lipoprotein, acetate, and acetic acid.

11. The method according to claim 1 or 2, wherein the sample is a blood serum sample and the metabolites are four or more (e.g. five or more, six or more or seven or more) metabolites selected from: myo-inositol, glucose, β-hydroxybutyrate, an N-acetylated glycoprotein (NAC), threonine, a lipoprotein, lactate, and acetate.

12. The method according to claim 1 or 2, wherein the sample is a blood serum sample and the metabolites are four or more (e.g. five or more, six or more or seven or more) metabolites selected from: myo-inositol, glucose, β-hydroxybutyric acid, an N-acetylated glycoprotein (NAC), threonine, a lipoprotein, lactic acid, and acetic acid.

13. The method according to any one of the preceding claims, further comprising comparing a concentration of citrate and/or citric acid present in a sample obtained from a subject with the concentration of citrate and/or citric acid, respectively, in a reference standard; and
diagnosing cancer when:
(i) the concentration of citrate and/or citric acid is higher in the sample relative to the reference standard, wherein the reference standard is a non-cancer reference standard; and/or
(ii) the concentration of citrate and/or citric acid is the same or higher in the sample relative to the reference standard, wherein the reference standard is a cancer reference standard; or
not diagnosing cancer when:
(iii) the concentration of citrate and/or citric acid is the same or lower in the sample relative to the reference standard, wherein the reference standard is a non-cancer reference standard; and/or
(iv) the concentration of citrate and/or citric acid is lower in the sample relative to the reference standard, wherein the reference standard is a secondary cancer reference standard.

14. A method for diagnosing cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 0.80-0.86 ppm, 0.86-0.92 ppm, 1.15-1.30 ppm, 1.30-1.39 ppm, 1.53-1.61 ppm, 2.20-2.26 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 1.31-1.35 ppm, 4.08-4.14 ppm, and 1.88-1.93 ppm; and
(b) diagnosing cancer when:
(i) the intensity of one or more chemical shift region(s) selected from: 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, and 4.06-4.3 ppm is higher in the sample relative to the reference standard, wherein the reference standard is a non-cancer reference standard; and/or
(ii) the intensity of one or more chemical shift region(s) selected from: 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm is lower in the sample relative to the reference standard, wherein the reference standard is a non-cancer reference standard; and/or
(iii) the intensity of one or more chemical shift region(s) selected from: 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, and 4.06-4.3 ppm is the same or higher in the sample relative to the reference standard, wherein the reference standard is a cancer reference standard; and/or
(iv) the intensity of one or more chemical shift region(s) selected from: 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm is the same or lower in the sample relative to the reference standard, wherein the reference standard is a cancer reference standard; or
(c) not diagnosing cancer when:
(i) the intensity of one or more chemical shift region(s) selected from: 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, and 4.06-4.3 ppm is the same or lower in the sample relative to the reference standard, wherein the reference standard is a non-cancer reference standard; and/or
(ii) the intensity of one or more chemical shift region(s) selected from: 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm is the same or higher in the sample relative to the reference standard, wherein the reference standard is a non-cancer reference standard; and/or
(iii) the intensity of one or more chemical shift region(s) selected from: 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, and 4.06-4.3 ppm is lower in the sample relative to the reference standard, wherein the reference standard is a cancer reference standard; and/or
(iv) the intensity of one or more chemical shift region(s) selected from: 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm is higher in the sample relative to the reference standard, wherein the reference standard is a cancer reference standard.

15. The method according to claim 2 or 14, wherein the chemical shift regions are at least:
(a) one or more (e.g. two or more, three or more, four or more or five or more) chemical shift regions(s) selected from: 0.80-0.86 ppm, 0.86-0.92 ppm, 1.15-1.30 ppm, 1.30-1.39 ppm, 1.53-1.61 ppm, and/or 2.20-2.26 ppm; and
(b) one or more (e.g. two or more, three or more, four or more, five or more, six or more, seven or more or eight or more) chemical shift regions(s) selected from: 1.19-1.21 ppm, 2.27-2.45 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm.

16. The method according to claim 2 or 14, wherein the chemical shift regions are at least:
(a) 1.19-1.21 ppm and/or 2.27-2.45 ppm; and
(b) two or more (e.g. three or more, four or more, five or more, six or more, seven or more, eight or more, nine or more, ten or more, eleven or more, twelve or more, thirteen or more, fourteen or more, fifteen or more or sixteen or more) chemical shift regions selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm.

17. The method according to claim 2 or 14, wherein the chemical shift regions are at least:
(a) 1.19-1.21 ppm and/or 2.27-2.45 ppm; and
(b) three or more (e.g. four or more, five or more, six or more, seven or more, eight or more, nine or more, ten or more, eleven or more, twelve or more, thirteen or more, fourteen or more, fifteen or more or sixteen or more, seventeen or more, eighteen or more or nineteen or more) chemical shift regions selected from: 3.17-3.95 ppm, 4.63-4.66 ppm, 5.22-5.25 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm.

18. The method according to claim 2 or 14, wherein the chemical shift regions are at least:
(a) 1.93-2.10 ppm; and
(b) one or more (e.g. two or more, three or more, four or more, five or more, six or more, seven or more, eight or more, nine or more, ten or more, eleven or more, twelve or more, thirteen or more, fourteen or more, fifteen or more or sixteen or more, seventeen or more, eighteen or more, nineteen or more or twenty or more) chemical shift region(s) selected from: 3.17-3.95 ppm, 4.63-4.66 ppm, 5.22-5.25 ppm, 1.19-1.21 ppm, 2.27-2.45 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm.

19. The method according to claim 2 or 14, wherein the biofluid sample is a blood sample (preferably a serum sample) and wherein the one or more (e.g. two or more or three or more) chemical shift regions(s) comprise at least 3.63-3.65 ppm, 3.53-3.58 ppm, 3.93-3.98 ppm, and/or 3.25-3.29 ppm.

20. The method according to claim 2 or 14, wherein the sample is a blood serum sample and the one or more (e.g. two or more, three or more, four or more, five or more or six or more) chemical shift regions(s) comprise at least 0.80-0.86 ppm, 0.86-0.92 ppm, 1.15-1.30 ppm, 1.30-1.39 ppm, 1.53-1.61 ppm, and/or 2.20-2.26 ppm.

21. The method according to claim 2 or 14, wherein the sample is a blood serum sample and the one or more chemical shift regions(s) comprise at least 1.88-1.93 ppm.

22. The method according to claim 2 or 14, wherein the sample is a blood serum sample and the one or more (e.g. two or more, three or more, four or more, five or more, six or more, seven or more, eight or more, nine or more or ten or more) chemical shift regions(s) are selected from: 3.63-3.65 ppm, 3.53-3.58 ppm, 3.93-3.98 ppm, 3.25-3.29 ppm, 0.80-0.86 ppm, 0.86-0.92 ppm, 1.15-1.30 ppm, 1.30-1.39 ppm, 1.53-1.61 ppm, 2.20-2.26 ppm, and 1.88-1.93 ppm.

23. The method according to claim 2 or 14, wherein the sample is a blood serum sample and the chemical shift regions are four or more (e.g. five or more, six or more, seven or more, eight or more, nine or more, ten or more, eleven or more, twelve or more, thirteen or more, fourteen or more, fifteen or more or sixteen or more, seventeen or more, eighteen or more, nineteen or more, twenty or more or twenty one or more) chemical shift regions selected from: 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 1.19-1.21 ppm, 2.27-2.45 ppm, 1.93-2.10 ppm, 3.57-3.59 ppm, 1.30-1.35 ppm, 4.06-4.3 ppm, 0.80-0.86 ppm, 0.86-0.92 ppm, 1.15-1.30 ppm, 1.30-1.39 ppm, 1.53-1.61 ppm, 2.20-2.26 ppm, 1.31-1.35 ppm, 4.08-4.14 ppm, and 1.88-1.93 ppm.

24. The method according to any one of claim 2 or 14-23, further comprising comparing an intensity of a chemical shift region of a ¹H-NMR spectrum of a sample obtained from a subject with the intensity of the same one or more chemical shift region of a ¹H-NMR reference standard, wherein the chemical shift region is 2.51-2.70 ppm; and
diagnosing cancer when:
(i) the intensity of the chemical shift region of the ¹H-NMR spectrum of the sample obtained from the subject is higher in the sample relative to the reference standard, wherein the reference standard is a non-cancer reference standard; and/or
(ii) the intensity of the chemical shift region of the ¹H-NMR spectrum of the sample obtained from the subject is the same or higher in the sample relative to the reference standard, wherein the reference standard is a cancer reference standard; or
not diagnosing cancer when:
(iii) the intensity of the chemical shift region of the ¹H-NMR spectrum of the sample obtained from the subject is the same or lower in the sample relative to the reference standard, wherein the reference standard is a non-cancer reference standard; and/or
(iv) the intensity of the chemical shift region of the ¹H-NMR spectrum of the sample obtained from the subject is lower in the sample relative to the reference standard, wherein the reference standard is a secondary cancer reference standard.

25. The method according to any one of claim 2 or 14-24, further comprising comparing an intensity of a chemical shift region of a ¹H-NMR spectrum of a sample obtained from a subject with the intensity of the same one or more chemical shift region of a ¹H-NMR reference standard, wherein the chemical shift region is 3.17-3.31 ppm; and
diagnosing a secondary cancer when:
(i) the intensity of the chemical shift region of the ¹H-NMR spectrum of the sample obtained from the subject is higher in the sample relative to the reference standard, wherein the reference standard is a non-cancer reference standard; and/or
(ii) the intensity of the chemical shift region of the ¹H-NMR spectrum of the sample obtained from the subject is the same or higher in the sample relative to the reference standard, wherein the reference standard is a secondary cancer reference standard; or
diagnosing a primary cancer when:
(iii) the intensity of the chemical shift region of the ¹H-NMR spectrum of the sample obtained from the subject is lower in the sample relative to the reference standard, wherein the reference standard is a non-cancer reference standard; and/or
(iv) the intensity of the chemical shift region of the ¹H-NMR spectrum of the sample obtained from the subject is the same or lower in the sample relative to the reference standard, wherein the reference standard is a primary cancer reference standard.

26. The method according to any one of the preceding claims, wherein the reference standard is a non-cancer reference standard.

27. The method according to any one of the preceding claims, wherein the non-cancer reference standard is representative of a subject that does not have (and optionally has not had) cancer but that has one or more symptoms of ill health.

28. The method according to any one of the preceding claims, wherein the non-cancer reference standard is representative of a subject that does not have (and optionally has not had) cancer but that has one or more non-specific symptoms/signs, preferably wherein said one or more non-specific symptoms/signs include one or more of: unexplained weight loss, severe unexplained fatigue, persistent nausea or appetite loss, new atypical pain, an unexplained laboratory test finding, and/or GP clinical suspicion of cancer or serious disease (GP ‘gut feeling’).

29. A method for diagnosing a primary cancer or a secondary cancer, the method comprising:
(a) comparing a concentration of one or more metabolite(s) comprised in a sample obtained from a subject with the concentration of the same one or more metabolite(s) in a reference standard, wherein the one or more metabolite(s) are selected from: β-hydroxybutyrate, β-hydroxybutyric acid, a lipoprotein, an N-acetylated glycoprotein (NAC), threonine, myo-inositol, glucose, lactate, lactic acid, acetate, acetic acid, and free choline; and
(b) diagnosing a secondary cancer when:
(i) the concentration of one or more metabolite(s) selected from: β-hydroxybutyrate, β-hydroxybutyric acid, a lipoprotein, lactate, lactic acid, acetate, acetic acid, and free choline is higher in the sample relative to the reference standard, wherein the reference standard is a primary cancer reference standard; and/or
(ii) the concentration of one or more metabolite(s) selected from: NAC, threonine, myo-inositol, and glucose is lower in the sample relative to the reference standard, wherein the reference standard is a primary cancer reference standard; and/or
(iii) the concentration of one or more metabolite(s) selected from: β-hydroxybutyrate, β-hydroxybutyric acid, a lipoprotein, lactate, lactic acid, acetate, acetic acid, and free choline is the same or higher in the sample relative to the reference standard, wherein the reference standard is a secondary cancer reference standard; and/or
(iv) the concentration of one or more metabolite(s) selected from: NAC, threonine, myo-inositol, and glucose is the same or lower in the sample relative to the reference standard, wherein the reference standard is a secondary cancer; or
(c) diagnosing a primary cancer when:
(i) the concentration of one or more metabolite(s) selected from: β-hydroxybutyrate, β-hydroxybutyric acid, a lipoprotein, lactate, lactic acid, acetate, acetic acid, and free choline is the same or lower in the sample relative to the reference standard, wherein the reference standard is a primary cancer reference standard; and/or
(ii) the concentration of one or more metabolite(s) selected from: NAC, threonine, myo-inositol, and glucose is the same or higher in the sample relative to the reference standard, wherein the reference standard is primary cancer reference standard; and/or
(iii) the concentration of one or more metabolite(s) selected from: β-hydroxybutyrate, β-hydroxybutyric acid, a lipoprotein, lactate, lactic acid, acetate, acetic acid, and free choline is lower in the sample relative to the reference standard, wherein the reference standard is a secondary cancer reference standard; and/or
(iv) the concentration of one or more metabolite(s) selected from: NAC, threonine, myo-inositol, and glucose is higher in the sample relative to the reference standard, wherein the reference standard is a secondary cancer reference standard.

30. A method for diagnosing a primary cancer or a secondary cancer, the method comprising:
(a) comparing a concentration of one or more metabolite(s) comprised in a sample obtained from a subject with the concentration of the same one or more metabolite(s) in a reference standard (preferably a cancer reference standard), wherein the one or more metabolite(s) are selected from: β-hydroxybutyrate, β-hydroxybutyric acid, a lipoprotein, an N-acetylated glycoprotein (NAC), threonine, myo-inositol, glucose, lactate, lactic acid, acetate, acetic acid, and free choline; or
(b) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 1.19-1.21 ppm, 2.27-2.45 ppm, 0.86-0.92 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm; and
(c) diagnosing a primary cancer or a secondary cancer based on the comparison.

31. The method according to claim 29 or 30, wherein the one or more metabolite(s) are one or more (e.g. two or more, three or more, four or more, five or more or six or more) metabolite(s) selected from: β-hydroxybutyrate, β-hydroxybutyric acid, an N-acetylated glycoprotein (NAC), threonine, myo-inositol, glucose, and free choline.

32. The method according to claim 29 or 30, wherein the metabolites are four or more (e.g. five or more, six or more, seven or more, eight or more or nine or more) metabolites selected from: β-hydroxybutyrate, β-hydroxybutyric acid, a lipoprotein, an N-acetylated glycoprotein (NAC), threonine, myo-inositol, glucose, lactate, acetate, and free choline.

33. The method according to claim 29 or 30, wherein the metabolites are four or more (e.g. five or more, six or more, seven or more, eight or more or nine or more) metabolites selected from: β-hydroxybutyrate, β-hydroxybutyric acid, a lipoprotein, an N-acetylated glycoprotein (NAC), threonine, myo-inositol, glucose, lactic acid, acetic acid, and free choline.

34. The method according to any one of claims 29-33, further comprising comparing a concentration of citrate and/or citric acid present in a sample obtained from a subject with the concentration of citrate and/or citric acid, respectively, in a reference standard; and
diagnosing a secondary cancer when:
(i) the concentration of citrate and/or citric acid is lower in the sample relative to the reference standard, wherein the reference standard is a primary cancer reference standard; and/or
(ii) the concentration of citrate and/or citric acid is the same or lower in the sample relative to the reference standard, wherein the reference standard is a secondary cancer; or
diagnosing a primary cancer when:
(i) the concentration of citrate and/or citric acid is the same or higher in the sample relative to the reference standard, wherein the reference standard is primary cancer reference standard; and/or
(ii) the concentration of citrate and/or citric acid is higher in the sample relative to the reference standard, wherein the reference standard is a secondary cancer reference standard.

35. A method for diagnosing a primary cancer or a secondary cancer, the method comprising:
(a) comparing an intensity of one or more chemical shift region(s) of a ¹H-NMR spectrum of a sample obtained from a subject with the intensity of the same one or more chemical shift region(s) of a ¹H-NMR reference standard, wherein the one or more chemical shift region(s) are selected from: 1.19-1.21 ppm, 2.27-2.45 ppm, 0.86-0.92 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm; and
(b) diagnosing a secondary cancer when:
(i) the intensity of one or more chemical shift region(s) selected from: 1.19-1.21 ppm, 2.27-2.45 ppm, 0.86-0.92 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is higher in the sample relative to the reference standard, wherein the reference standard is a primary cancer reference standard; and/or
(ii) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, and 3.17-3.95 ppm, 4.63-4.66 ppm, and 5.22-5.25 ppm is lower in the sample relative to the reference standard, wherein the reference standard is a primary cancer reference standard; and/or
(iii) the intensity of one or more chemical shift region(s) selected from: 1.19-1.21 ppm, 2.27-2.45 ppm, 0.86-0.92 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or higher in the sample relative to the reference standard, wherein the reference standard is a secondary cancer reference standard; and/or
(iv) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or lower in the sample relative to the reference standard, wherein the reference standard is a secondary cancer; or
(c) diagnosing a primary cancer when:
(i) the intensity of one or more chemical shift region(s) selected from: 1.19-1.21 ppm, 2.27-2.45 ppm, 0.86-0.92 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is the same or lower in the sample relative to the reference standard, wherein the reference standard is a primary cancer reference standard; and/or
(ii) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is the same or higher in the sample relative to the reference standard, wherein the reference standard is primary cancer reference standard; and/or
(iii) the intensity of one or more chemical shift region(s) selected from: 1.19-1.21 ppm, 2.27-2.45 ppm, 0.86-0.92 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm is lower in the sample relative to the reference standard, wherein the reference standard is a secondary cancer reference standard; and/or
(iv) the intensity of one or more chemical shift region(s) selected from: 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, and 5.22-5.25 ppm is higher in the sample relative to the reference standard, wherein the reference standard is a secondary cancer reference standard.

36. The method according to claim 30 or 35, wherein the one or more chemical shift regions(s) are one or more (e.g. two or more, three or more, four or more, five or more, six or more, seven or more, eight or more, nine or more, ten or more, eleven or more, twelve or more or thirteen or more) chemical shift regions(s) selected from: 1.19-1.21 ppm, 2.27-2.45 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, and 3.17-3.31 ppm.

37. The method according to claim 30 or 35, wherein the chemical shift regions are six or more (e.g. seven or more, eight or more, nine or more, ten or more, eleven or more, twelve or more, thirteen or more, fourteen or more, fifteen or more or sixteen or more, seventeen or more, eighteen or more, nineteen or more, twenty or more, twenty one or more or twenty two or more) chemical shift regions selected from: 1.19-1.21 ppm, 2.27-2.45 ppm, 0.86-0.92 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, 3.25-3.29 ppm, 3.17-3.95 ppm, 4.63-4.66 ppm, 5.22-5.25 ppm, 0.80-0.86 ppm, 1.15-1.30 ppm, 1.31-1.35 ppm, 4.08-4.14 ppm, 1.30-1.39 ppm, 1.53-1.61 ppm, 1.88-1.93 ppm, 2.20-2.26 ppm, and 3.17-3.31 ppm.

38. The method according to any one of claim 30 or 35-37, further comprising comparing an intensity of a chemical shift region of a ¹H-NMR spectrum of a sample obtained from a subject with the intensity of the same one or more chemical shift region of a ¹H-NMR reference standard, wherein the chemical shift region is 2.51-2.70 ppm; and
diagnosing a secondary cancer when:
(i) the intensity of the chemical shift region of the ¹H-NMR spectrum of the sample obtained from the subject is lower in the sample relative to the reference standard, wherein the reference standard is a primary cancer reference standard; and/or
(ii) the intensity of the chemical shift region of the ¹H-NMR spectrum of the sample obtained from the subject is the same or lower in the sample relative to the reference standard, wherein the reference standard is a secondary cancer; or
diagnosing a primary cancer when:
(iii) the intensity of the chemical shift region of the ¹H-NMR spectrum of the sample obtained from the subject is the same or higher in the sample relative to the reference standard, wherein the reference standard is primary cancer reference standard; and/or
(iv) the intensity of the chemical shift region of the ¹H-NMR spectrum of the sample obtained from the subject is higher in the sample relative to the reference standard, wherein the reference standard is a secondary cancer reference standard.

39. The method according to any one of the preceding claims, wherein the method further comprises measuring a concentration of one or more metabolite(s) present in a sample obtained from a subject.

40. The method according to any one of the preceding claims, wherein the sample is a biofluid sample, preferably wherein the sample is a blood sample.

41. The method according to any one of the preceding claims, wherein the sample is a blood serum sample.

42. The method according to any one of claim 1-13, 26-34, or 39-41. wherein the concentration of the one or more metabolites has been, or is, determined using: nuclear magnetic resonance (NMR) spectroscopy, mass spectrometry, HPLC-UV, infrared spectrometry, a biochemical assay or a combination thereof.

43. The method according to any one of claim 1-13, 26-34, or 39-42, wherein the concentration of the one or more metabolites has been, or is, determined using ¹H-NMR spectroscopy.

44. The method according to any one of claims 2, 14-28, 30, and 35-43, wherein the chemical shift region(s) is/are reported relative to lactate —CH₃ referenced at 1.33 ppm.

45. The method according to any one of the preceding claims, wherein the sample comprises D₂O.

46. The method according to any one of the preceding claims, wherein the method comprises comparing the concentration two or more metabolites or comparing the intensity of two or more chemical shift regions, respectively.

47. The method according to any one of the preceding claims, wherein the method comprises comparing the concentration three or more metabolites or comparing the intensity of three or more chemical shift regions, respectively.

48. The method according to any one of claim 1-28 or 39-47, wherein cancer is not diagnosed when:
(a) the concentration of one or more metabolite(s) selected from: glucose, NAC, threonine, and myo-inositol is lower in the sample relative to the reference standard, wherein the reference standard is a secondary cancer reference standard; and/or
(b) the concentration of β-hydroxybutyrate and/or β-hydroxybutyric acid, is lower in the sample relative to the reference standard, wherein the reference standard is a primary cancer reference standard; and/or
(c) the concentration of one or more metabolite(s) selected from: a lipoprotein, lactate, lactic acid, acetate, and acetic acid is higher in the sample relative to the reference standard, wherein the reference standard is a secondary cancer reference standard; and/or
(d) the intensity of one or more chemical shift region(s) selected from: 3.17-3.95 ppm, 4.63-4.66 ppm, 5.22-5.25 ppm, 1.93-2.10 ppm, 3.57-3.59 ppm, 1.30-1.35 ppm, 4.06-4.3 ppm, 3.63-3.65 ppm, 3.53-3.58 ppm, 3.93-3.98 ppm, and 3.25-3.29 ppm is lower in the sample relative to the reference standard, wherein the reference standard is a secondary cancer reference standard; and/or
(e) the intensity of one or more chemical shift region(s) selected from: 1.19-1.21 ppm and 2.27-2.45 ppm is lower in the sample relative to the reference standard, wherein the reference standard is a primary cancer reference standard; and/or
(f) the intensity of one or more chemical shift region(s) selected from: 0.80-0.86 ppm, 0.86-0.92 ppm, 1.15-1.30 ppm, 1.31-1.35 ppm, 4.08-4.14 ppm, 1.30-1.39 ppm, 1.53-1.61 ppm, 1.88-1.93 ppm, and 2.20-2.26 ppm is higher in the sample relative to the reference standard, wherein the reference standard is a secondary cancer reference standard.

49. The method according to any one of the preceding claims, wherein the subject is a subject that has presented with one or more symptoms of ill health.

50. The method according to any one of the preceding claims, wherein the subject is a subject that has presented with one or more non-specific symptoms/signs, preferably wherein said one or more non-specific symptoms/signs include one or more of: unexplained weight loss, severe unexplained fatigue, persistent nausea or appetite loss, new atypical pain, an unexplained laboratory test finding, and/or GP clinical suspicion of cancer or serious disease (GP ‘gut feeling’).

51. The method according to any one of the preceding claims, wherein the metabolite(s) is/are (a) blood metabolite(s).

52. The method according to any one of the preceding claims, wherein the metabolite(s) is/are (a) serum metabolite(s).

53. The method according to any one of the preceding claims, wherein the cancer reference standard (e.g. the primary cancer reference standard and/or secondary cancer reference standard) is representative of a subject that has cancer or has had cancer (e.g. primary or secondary cancer) (preferably that has cancer, e.g. primary or secondary cancer) and has one or more symptoms of ill health.

54. The method according to any one of the preceding claims, wherein the cancer reference standard (e.g. the primary cancer reference standard and/or secondary cancer reference standard) is representative of a subject that has cancer or has had cancer (e.g. primary or secondary cancer) (preferably that has cancer, e.g. primary or secondary cancer) and has one or more non-specific symptoms/signs, preferably wherein said one or more non-specific symptoms/signs include one or more of: unexplained weight loss, severe unexplained fatigue, persistent nausea or appetite loss, new atypical pain, an unexplained laboratory test finding, and/or GP clinical suspicion of cancer or serious disease (GP ‘gut feeling’).

55. The method according to any one of the preceding claims further comprising recording the output of at least one step on a data-storage medium.

56. A data-storage medium comprising data obtained by the method of any one of the preceding claims.

57. A computer program product comprising program instructions to cause a processor to perform the method of any one of claims 1-55.

58. A method of treating cancer, the method comprising:
(a) obtaining the results of a method according to any one of claims 1-55; and
(b) administering a cancer therapy when a cancer is diagnosed; and
(c) optionally administering a different therapy when the cancer is not diagnosed.
